(19) United States
(12) Patent Application Publication
(10) Pub. No.: US 2003/0170743 A1
(43) Pub. Date: Sep. 11, 2003

(54) Polynucleotides and polypeptides associated with the development of osteoarthritis

(76) Inventors: Julie Carman, Lawrenceville, NJ (US); Steven G. Nadler, Princeton, NJ (US); Michael Bowen, Rockville, MD (US); Michael G. Neubauer, Skillman, NJ (US); Pin Lu, Cherry Hill, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/308,448

(22) Filed: Dec. 3, 2002

Related U.S. Application Data
(60) Provisional application No. 60/337,417, filed on Dec. 3, 2001.

(51) Int. Cl. G01N 33/53; G01N 33/567

(52) U.S. Cl. 435/7.2

(57) ABSTRACT

The present invention is directed to polynucleotides encoding polypeptides associated with the development of osteoarthritis and homologs thereof. The invention further relates to diagnostic and therapeutic methods for utilizing these polynucleotides and polypeptides in the diagnosis, treatment, and/or prevention of osteoarthritis and related disease states. The invention further relates to screening methods for identifying agonists and antagonists of the polynucleotides and polypeptides of the present invention, and compounds identified thereby.

### CROSS-REFERENCE TO RELATED APPLICATIONS

This application claims priority to U.S. Provisional Patent Application No. 60/337,417, filed Dec. 3, 2001, and hereby expressly incorporated by reference in its entirety

### FIELD OF THE INVENTION

The present invention provides polynucleotides encoding polypeptides associated with the development of osteoarthritis and homologs thereof. Also provided are vectors, host cells, antibodies, and recombinant and synthetic methods for producing said polypeptides. The invention further relates to diagnostic and therapeutic methods for utilizing these polypeptides in the diagnosis, treatment, and/or prevention of osteoarthritis and related disease states. The invention further relates to screening methods for identifying agonists and antagonists of the polynucleotides and polypeptides of the present invention.

### BACKGROUND OF RELATED TECHNOLOGY

Osteoarthritis (OA) is a chronic, degenerative disease characterized by progressive loss of articular cartilage. This loss is accompanied by changes in the bone underneath the cartilage including development of marginal outgrowths and thickening of the bony envelope. The disease affects the entire joint structure including the synovial membrane, subchondral bone, ligaments, and periarticular muscles. Many patients exhibit signs of local inflammation including synovial hypertrophy and hyperplasia accompanied by an infiltration of the sublining tissue with a mixed population of inflammatory cells. The inflammation is thought to be secondary to the destruction of cartilage and release of cartilage breakdown products in synovial fluid. Many of these products activate synovial cells to produce proinflammatory cytokines including interleukin-1 (IL-1) and TNF-α. Increased production of IL-1α, IL-1β, and TNF-α has been observed in OA synovial membranes. (Smith, et al., J. Rheumatol. 24:365-371 (1997)).

The primary cell type involved in the pathology of OA is the chondrocyte. Chondrocytes represent the major cellular component of cartilage, and are involved in maintaining the cartilage matrix. The components of the extracellular matrix secreted by chondrocytes include highly crosslinked fibrils of triple helical type II collagen molecules, the proteoglycan aggrecan, and other collagenous and noncollagenous matrix proteins. Mechanical stress is thought to initiate the cartilage lesion by disrupting chondrocyte-matrix associations and altering metabolic responses in the chondrocyte. In response to this stress, and to the cytokines produced by synovial cells, chondrocytes synthesize a number of cytokines and inflammatory mediators including IL-1β, TNF-α, IL-6, IL-8, nitric oxide, and prostaglandins, as well as matrix degrading enzymes including matrix metalloproteinases (MMPs), aggrecanase, plasminogen activator, and cathepsins. Nitric oxide promotes cartilage catabolism via several mechanisms including inhibition of cartilage proteoglycan synthesis (Taskiran, et al., Biochem. Biophys. Res. Commun. 200:142-148 (1994), and stimulation of MMP activity (Murrell, et al., Biochem. Biophys. Res. Commun. 206:15-21 (1995)).

The cytokines IL-1β and TNF-α are thought to play a direct role in the pathophysiology of OA. In animal models of OA, intraarticular injection of recombinant IL-1 induced cartilage degradation. Co-injection with TNF-α induced more severe damage than injection of either cytokine alone (Goldring, Arthritis Rheum. 43:1916-1926 (2000)). Conversely, in a dog OA model, intraarticular injection of IL-1 receptor antagonist, a natural inhibitor of IL-1, was chondroprotective and suppressed expression of the MMPs collagenase-1 and stromelysin-1 in cartilage and synovium (Caron, et al., Arthritis Rheum. 39:1535-1544 (1996)). These cytokines act in an autocrine manner to induce their own synthesis, as well as the synthesis of other cytokines such as IL-8, IL-6, and leukocyte inhibitory factor (LIF) which mediate cartilage catabolic responses such as the stimulation of cartilage proteoglycan resorption.

A major mechanism by which IL-1 and TNF-α contribute to cartilage damage is via induction of MMPs that degrade cartilage collagens and proteoglycans. The proteases involved in type II collagen degradation include MMPs 1, 8, 13, and 14 (Billinghurst, et al., J. Clin. Invest. 99:1534-1545 (1997)). MMP-13 has been detected in OA cartilage, and degrades type II collagen more efficiently than many other collagenases, suggesting that it may play a major role in cartilage degradation (Mitchell, et al., J. Clin. Invest. 97:761-768 (1996)). Other proteases involved in cartilage degradation include stromelysins (MMP-3, -10, -11) that degrade proteoglycans; gelatinases (MMP-2, -9); cathepsins B, D, G, L; plasminogen activators; and aggrecanases (ADAM-TS4, ADAM-TS5).

Several genetic studies have identified mutations that predispose individuals to OA. The majority of heritable cases of OA are due to monogenic mutations in the type II collagen gene which can lead to premature OA (Mundlos, et al., FASEB J. 11:227-233 (1997)). Mutations in other cartilage-specific collagens have also been associated with secondary OA in humans (Mundlos et al., FASEB J. 11:227-233 (1997)). Mutations in the α1 and α2 chains of type IX collagen are associated with changes in knee joint cartilage in families with multiple epiphyseal dysplasia (Muragaki et al., Nature Genet. 12:103-105 (1996)). A polymorphism in the region of the aggrecan gene that encodes the chondroitin sulfate attachment sites was associated with bilateral hand OA (Horton Jr., et al., Osteoarthritis Cartilage 6:245-251 (1998)).

Other candidate genes for the common forms of OA include the vitamin D receptor gene, the insulin-like growth factor I gene, cartilage oligomeric protein genes, and the HLA region. The gene for the vitamin D receptor maps near the gene encoding type II collagen. A Taq 1 polymorphism in the vitamin D receptor locus was associated with early knee OA in women (Keen, et al., Arthritis Rheum. 40:1444-1449 (1997); Uitterlinden, et al., Arthritis Rheum. 43:1456-1464 (2000)). Polymorphisms in the insulin-like growth factor I gene were associated with the presence of radiological OA (Meulenbelt, et al., Ann. Rheum. Dis. 57:371-374 (1998)). Mutations in the gene encoding cartilage oligomeric matrix protein have been shown to cause pseudoachondroplasia and multiple epiphyseal dysplasia that are characterized by early onset OA (Deere, et al., Am. J. Med. Genet. 85:486-490 (1999)). Expression of HLA-DR4 was associated with congenital dislocation of the hip joint and progressive dysplastic OA of the hip (Torisu, et al., Arthritis Rheum. 36:815-818 (1993)). Other linkage studies have identified loci on chromosomes 2q, 4, 6, 7, 11q, 16, and X that increase susceptibility to OA (Loughlin, Curr. Opin. Rheumatol. 13:111-116 (2001)).

A number of biomarkers of cartilage turnover have also been identified. These include type II collagen degradation products, cartilage oligomeric matrix protein, and two epitopes of aggrecan (Garnero, et al., Ann. Rheum. Dis. 60:545-548 (2001)). Gene expression analysis has also been used to identify OA biomarkers. In one study, using real time PCR, increases in osteopontin levels were detected in cartilage samples from OA patients relative to normal controls. The mRNA ratios of collagen type II to I and of aggrecan to versican, defined as indexes of chondrocyte differentiation, were significantly higher in the control as compared to OA samples (Martin, et al., Osteoarthritis Cartilage 9:112-118 (2001)).

Accordingly, there is a continuing need to identify genes whose expression is associated with the development of OA. The identification of such genes permits the development of clones expressing such genes, thereby permitting the identification of compounds capable of modulating the activity of such genes and/or their expression products. Such compounds may have therapeutic utility in the diagnosis and/or treatment of OA and related disease states. The present invention is directed to meeting these and other needs.

### BRIEF DESCRIPTION OF THE DRAWINGS

FIGS. 1, 3, 6, 9, 12, 15, 18, 21, 24, 27, 30, 33, 35, 38, 41 and 44 show microarray data for genes of the present invention shown to be upregulated and downregulated in osteoarthritis synovial fluid.

FIGS. 2, 4, 7, 10, 13, 16, 19, 22, 25, 28, 31, 3, 36, 39, 42 and 45 show polynucleotide sequences for genes of the present invention shown to be upregulated and downregulated in osteoarthritis synovial fluid.

FIGS. 5, 8, 11, 14, 17, 20, 23, 26, 29, 32, 37, 40, 43 and 46 show amino acid sequences for the expression product of genes of the present invention shown to be upregulated and downregulated in osteoarthritis synovial fluid.

### SUMMARY OF THE INVENTION

In one aspect, the present invention is directed to an assay for identifying a compound that modulates the activity of a gene associated with osteoarthritis, including the steps of: (1) providing a cell expressing a gene associated with osteoarthritis, wherein the nucleic acid sequence of the gene associated with osteoarthritis is at least 95% identical to a nucleic acid sequence selected from the group consisting of SEQ ID NO:7, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:31, SEQ ID NO:33, and SEQ ID NO:35; (2) contacting the cell expressing the gene associated with osteoarthritis with a test compound; and (3) determining whether the test compound modulates the activity of the gene associated with osteoarthritis. The assay may be cell-based assay or may be a cell-free assay, such as a ligand-binding assay. The test compound desirably modulates the activity of the gene associated with osteoarthritis, may be an antagonist or an agonist of the gene associated with osteoarthritis, and may bind to the gene associated with osteoarthritis. The assay is desirably useful for identifying compounds which are useful for the treatment of osteoarthritis.

In another aspect, the present invention is directed to an assay for identifying a compound that modulates the activity of a protein associated with osteoarthritis, including the steps of: (1) providing a cell expressing a gene associated with osteoarthritis, wherein the gene encodes a polypeptide having an amino acid sequence which is at least 95% identical to an amino acid sequence selected from the group consisting of SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:30, SEQ ID NO:32, SEQ ID NO:34, and SEQ ID NO:36; (2) contacting the cell expressing the gene associated with osteoarthritis with a test compound; and (3) determining whether the test compound modulates the activity of the protein associated with osteoarthritis. The test compound desirably modulates the activity of the protein associated with osteoarthritis, may be an antagonist or an agonist of the protein associated with osteoarthritis, and may bind to the protein associated with osteoarthritis. The assay is desirably useful for identifying compounds which are useful for the treatment of osteoarthritis.

In another aspect, the present invention is directed to a method for the treatment of osteoarthritis, including the steps of: (1) identifying a patient suffering from osteoarthritis; and (2) administering to the patient a therapeutically effective amount of a modulator of a gene associated with osteoarthritis, wherein the gene associated with osteoarthritis has a nucleic acid sequence which is at least 95% identical to a nucleic acid sequence selected from the group consisting of SEQ ID NO:7, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:31, SEQ ID NO:33, and SEQ ID NO:35. The patient is desirably identified as suffering from osteoarthritis by measuring the expression level of the gene associated with osteoarthritis in the patient. The modulator is desirably an antagonist of a gene associated with osteoarthritis.

In another aspect, the present invention is directed to a method for the treatment of osteoarthritis, including the steps of: (1) identifying a patient suffering from osteoarthritis; and (2) administering to the patient suffering from osteoarthritis a therapeutically effective amount of a modulator of a polypeptide associated with osteoarthritis, wherein the polypeptide associated with osteoarthritis has an amino acid sequence which is at least 95% identical to an amino acid sequence selected from the group consisting of SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:30, SEQ ID NO:32, SEQ ID NO:34, and SEQ ID NO:36. The patient is desirably identified as suffering from osteoarthritis by measuring the expression level of the polypeptide associated with osteoarthritis. The modulator is desirably an antagonist of a polypeptide associated with osteoarthritis.

In another aspect, the present invention is directed to a method for the prevention of osteoarthritis, including the steps of: (1) identifying a patient at risk for osteoarthritis; and (2) administering to the patient at risk for osteoarthritis a therapeutically effective amount of a modulator of a gene associated with osteoarthritis, wherein the gene associated with osteoarthritis has a nucleic acid sequence which is at least 95% identical to a nucleic acid sequence selected from the group consisting of SEQ ID NO:7, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:31, SEQ ID NO:33, and SEQ ID NO:35. The patient is desirably identified as being at risk for osteoarthritis by measuring the expression level of the gene associated with osteoarthritis in the patient.

In another aspect, the present invention is directed to a method for the prevention of osteoarthritis, including the steps of: (1) identifying a patient at risk for osteoarthritis; and (2) administering to the patient at risk for osteoarthritis a therapeutically effective amount of a modulator of a polypeptide associated with osteoarthritis, wherein the polypeptide associated with osteoarthritis has an amino acid sequence which is at least 95% identical to an amino acid sequence selected from the group consisting of SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:30, SEQ ID) NO:32, SEQ ID NO:34, and SEQ ID NO:36. The patient is desirably identified as being at risk for osteoarthritis by measuring the expression level of the polypeptide associated with osteoarthritis in the patient.

In another aspect, the present invention is directed to a compound useful for the treatment of osteoarthritis, wherein the compound is identified by: (1) providing a cell expressing a gene associated with osteoarthritis, wherein the gene associated with osteoarthritis has a nucleic acid sequence which is at least 95% identical to a nucleic acid sequence selected from the group consisting of SEQ ID NO:7, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:31, SEQ ID NO:33, and SEQ ID NO:35; (2) contacting the cell expressing the gene associated with osteoarthritis with the compound; and (3) determining whether the compound modulates the activity of the gene associated with osteoarthritis.

In another aspect, the present invention is directed to a compound useful for the treatment of osteoarthritis, wherein the compound is identified by: (1) providing a cell expressing a polypeptide associated with osteoarthritis, wherein the polypeptide associated with osteoarthritis has an amino acid sequence which is at least 95% identical to an amino acid sequence selected from the group consisting of SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:30, SEQ ID NO:32, SEQ ID NO:34, and SEQ ID NO:36; (2) contacting the cell expressing the polypeptide associated with osteoarthritis with the compound; and (3) determining whether the compound modulates the activity of the polypeptide associated with osteoarthritis.

### DETAILED DESCRIPTION OF THE INVENTION

The present invention is directed to the identification of genes associated with OsteoArthritis (OA). Such genes and their polypeptide expression products are hereinafter referred to as “OA-associated genes and polypeptides”. In the present invention, OA-associated genes and polypeptides have been identified by probing Affymetrix chips with mRNA derived from the synovia of OA patients, as set forth in “Materials and Methods”, hereinbelow. Gene expression patterns were compared to those obtained using mRNA derived from synovia of control joint trauma patients. Several genes were identified as having significantly increased expression in the OA synovium relative to the controls, as further described hereinbelow.

The present invention provides synthetic methods for producing OA-associated genes and polypeptides. Also provided are diagnostic methods for detecting diseases, disorders, and/or conditions related to OA-associated genes and polypeptides, and therapeutic methods for treating such diseases, disorders, and/or conditions. The invention further relates to screening methods for identifying binding partners of OA-associated genes and polypeptides.

Functional assays useful in the present invention include LPS or IL-1α-induced expression of MMP-1, MMP-3, MMP-13, Cox-2 expression in bovine chondrocytes; LPS-induced TNFα and TNFα-induced IL-1β secretion by THP-1 monocytes; anti-CD3/anti-CD28-induced IL-2 secretion by Jurkat T cells; TNFα-induced IL-1β secretion by synovial fibroblasts; TNFα-induced E-selectin expression by endothelial cells; and anti-CD40-induced homotypic aggregation of Raji B cells.

One of skill in the art will recognize that OA-associated genes and polypeptides of the present invention are desirably murine or human, but may be from any suitable organism. The genomic and protein sequences of OA-associated genes and polypeptides from these organisms are readily accessed via Genbank or The National Center for Biotechnology Information. Sequences in the Genbank database which correspond to Genbank Accession numbers or Genbank Identifiers provided herein are hereby expressly incorporated in their entirety.

Further, derivatives and homologues of OA-associated genes and polypeptides may be used in the present invention. For example, nucleic acid sequences of OA-associated genes of the present invention may be altered by substitutions, additions, or deletions that provide for functionally equivalent-conservative variants of such genes. Further, one or more amino acid residues within the amino acid sequence of OA-associated polypeptides can be substituted by another amino acid of similar properties, such as, for example, positively charged amino acids (arginine, lysine, and histidine); negatively charged amino acids (aspartate and glutamate); polar neutral amino acids; and non-polar amino acids.

Other conservative amino acid substitutions can be taken from the Table 1, below.

TABLE 1
Conservative amino acid replacements

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D- Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the present invention are those with modifications which increase protein stability; such analogs may contain, for example, one or more non-peptide bonds (which replace the peptide bonds) in the protein sequence. Also included are analogs that include residues other than naturally occurring L-amino acids, e.g., D-amino acids or non-naturally occurring or synthetic amino acids, e.g., β or γ amino acids.

OA-associated polypeptides of the present invention may be modified by, for example, phosphorylation, sulfation, acylation, or other protein modifications. They may also be modified with a label capable of providing a detectable signal, either directly or indirectly, including, but not limited to, radioisotopes and fluorescent compounds.

It will be apparent to one of skill in the art that conventional screening assays may be used in methods of the present invention for the identification of modulators of OA-associated genes and polypeptides.

In the present invention, techniques for screening large gene libraries may include cloning the gene library into replicable expression vectors, transforming appropriate cells with the resulting library of vectors, and expressing the genes under conditions for detection of a desired activity. Techniques known in the art are amenable to high throughput analysis for screening large numbers of sequences created, e.g., by random mutagenesis techniques. High throughput assays can be followed by secondary screens in order to identify further biological activities which will, e.g., allow one skilled in the art to differentiate agonists from antagonists. The type of a secondary screen used will depend on the desired activity that needs to be tested.

Drug screening assays are also provided in the present invention. By producing purified and recombinant forms of OA-associated genes and polypeptides of the present invention, or fragments thereof, one skilled in the art can use these to screen for drugs which are either agonists or antagonists of the normal cellular function or their role in cellular signaling. In one aspect, the assay evaluates the ability of a compound to modulate binding between OA-associated genes and polypeptides of the present invention and a naturally occurring ligand. The term “modulating” encompasses enhancement, diminishment, activation or inactivation of activity of OA-associated genes and polypeptides. Assays useful for identifying ligands to OA-associated genes and polypeptides of the present invention are encompassed herein. Such ligands include peptides, proteins, small molecules, and antibodies, which are capable of binding to OA-associated genes and polypeptides of the present invention and modulating their activity. A variety of assay formats may be used in the present invention and are known by those skilled in the art.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as primary screens in that they can be generated to permit rapid development and relatively easy detection of an alteration in a molecular target which is mediated by a test compound.

Compounds identified using assays, as discussed hereinabove, may be antagonists or agonists of OA-associated genes and polypeptides. These compounds are useful in modulating the activity of OA-associated genes and polypeptides and in treating disorders associated with OA-associated genes and polypeptides.

“Disorders associated with OA-associated genes and polypeptides” refers to any disorder or disease state in which OA-associated genes and polypeptides play a regulatory role in the metabolic pathway of that disorder or disease. As used herein, the term “treating” refers to the alleviation of symptoms of a particular disorder in a patient, the improvement of an ascertainable measurement associated with a particular disorder, or the prevention of a particular immune, inflammatory or cellular response.

A compound which acts as a modulator of OA-associated genes and polypeptides may be administered for therapeutic use as a raw chemical or may be the active ingredient in a pharmaceutical formulation. Such formulations of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Compounds of the present invention may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents.

Such compounds may, for example, be administered in a form suitable for immediate release or extended release. Immediate release or extended release may be achieved by the use of suitable pharmaceutical compositions comprising compounds of the present invention, or, particularly in the case of extended release, by the use of devices such as subcutaneous implants or osmotic pumps. Compounds of the present invention may also be administered liposomally.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art.

Compounds of the present invention may also be delivered through the oral cavity by sublingual and/or buccal administration. Molded tablets, compressed tablets or freeze-dried tablets are exemplary forms which may be used. Exemplary compositions include those formulating the compound(s) of the present invention with fast dissolving diluents such as mannitol, lactose, sucrose and/or cyclodextrins.

Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquify and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to disorders associated with OA-associated genes and polypeptides.

The compounds of the present invention may be employed alone or in combination with each other and/or other suitable therapeutic agents useful in the treatment of disorders associated with OA-associated genes and polypeptides.

In another aspect, the present invention relates to the use of an isolated nucleic acid in “antisense” therapy. As used herein, “antisense” therapy refers to administration or in situ generation of oligonucleotides or their derivatives which specifically hybridize under cellular conditions with the cellular mRNA and/or genomic DNA of OA-associated genes so as to inhibit expression of the proteins encoded by such genes, e.g., by inhibiting transcription and/or translation. In general, “antisense” therapy refers to the range of techniques generally employed in the art, and includes any therapy which relies on specific binding to oligonucleotide sequences.

Gene constructs useful in antisense therapy may be administered may be administered in any biologically effective carrier, e.g., any formulation or composition capable of effectively delivering a nucleic acid sequence to cells in vivo. Approaches include insertion of the subject gene in viral vectors including recombinant retroviruses, adenoviruses, adeno-associated viruses, and herpes simplex virus-1, or recombinant bacterial or eukaryotic plasmids. Viral vectors transfect cells directly; an advantage of infection of cells with a viral vector is that a large proportion of the targeted cells can receive the nucleic acid. Several viral delivery systems are known in the art and can be utilized by one practicing the present invention.

In addition to viral transfer methods, non-viral methods may also be employed. Most non-viral methods of gene transfer rely on normal mechanisms used by mammalian cells for the uptake and intracellular transport of macromolecules. Exemplary gene delivery systems of this type include liposomal derived systems, poly-lysine conjugates, and artificial viral envelopes. Nucleic acid sequences may also be introduced to cell(s) by direct injection of the gene construct or by electroporation.

In clinical settings, the gene delivery systems can be introduced into a patient by any of a number of methods, each of which is known in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells which produce the gene delivery system.

The following sections sets forth the materials and methods utilized in the present invention.

### MATERIALS AND METHODS

1. RNA Isolation

Human knee biopsy samples were homogenized in 3 ml TRIZOL® Reagent (Life Technologies, Rockville, Md.) and frozen in liquid nitrogen. The samples were thawed, one-third (1 ml) of the sample removed, mixed with 1 ml TRIZOL®, homogenized, and snap frozen in liquid nitrogen. Following a thaw, the samples were spun at 14,000 rpm for 10 minutes at 4° C. The supernatants were transferred to new microfuge tubes, extracted with chloroform, and precipitated with isopropanol overnight at −20° C. The RNA was pelleted by centrifugation at 14,000 rpm for 30 minutes. The supernatant was aspirated, and the samples washed two times with 75% ethanol. Following the last spin, the pellets were air-dried, and resuspended in 20 ul of ultra-pure RNase-free water. The RNA samples were further purified using Qiagen RNease mini columns (Qiagen Inc., Valencia Calif.) according to manufacturer's instructions. The RNA was eluted with 50 ul of RNase-free water.

2. Probe Preparation

The RNA was treated in a total reaction volume of 100 ul with RNase Inhibitor (Invitrogen Corp., Carlsbad, Calif.), DNase I (Ambion, Houston, Tex.) for 30 minutes at 37° C. The treated RNA was purified using Qiagen RNease mini columns according to the manufacturer's instructions. For the first strand CDNA synthesis, the RNA was incubated with T7-(dT)24 primer:

(5′GGCCAGTGAATTGTAATACGACTCACTATAGGGAGGCGGTTTTTTTTTTTTT TTTTTTTTTTT3′) (SEQ ID NO:1) for 10 minutes at 70° C., followed by one minute on ice. First strand buffer, DTT, dNTP and RNase were added, and the samples incubated for 2 minutes at 45° C. Superscript II reverse transcriptase (Invitrogen Corp, Carlsbad, Calif.) was added, and the samples incubated for an additional 60 minutes at 45° C.

For the second strand synthesis, the first strand cDNA was incubated with second strand buffer, dNTPs, E. coli ligase, E. coli RNase H, E. coli Polymerase I in a total volume of 150 ul for two hours at 16° C. T4 polymerase was added, and the incubation continued for an additional 5 minutes. Following this incubation, EDTA was added, and the samples placed on ice. The cDNA samples were extracted with phenol:chloroform:isoamyl alcohol and precipitated by addition of 0.5 volumes of 7.5 M ammonium acetate and 2.5 volumes of 100% ethanol. The samples were pelleted by a 30 minute room temperature spin at 12,000×g. The pelleted samples were washed with 0.5 ml 80% ethanol, spun for 10 minutes at 12,000×g, and air dried. The samples were resuspended in 12 ul RNase free water.

The cDNA was labeled using the Enzo Bio Array High Yield RNA transcript labeling kit (Enzo Therapeutics, Farmingdale, N.Y.). The cDNA was incubated with HY reaction buffer, biotin labeled NTP, DTT, RNase mix, and T7 DNA polymerase for six hours at 37° C. Unincorporated nucleotides were removed using Qiagen RNeasy columns according to manufacturer's instructions. The cRNA was fragmented by addition of fragmentation buffer, and incubated for 35 minutes at 95° C. The fragmented cRNA (0.05 mg/ml) was added to a hybridization solution master mix that included 0.1 mg/ml herring sperm DNA, 5 nM oligo B2, 1× standard curve pool, 0.5 mg/ml acetylated BSA, 1×MES hybridization buffer.

The Affymetrix human U95v2 A, B, and C chips were probed with the hybridization master mix. The hybridization, washing, and Phycoerythrin streptavidin staining were performed using the Affymetrix hybridization oven and fluidics workstation according to manufacturer's instructions. Stained chips were scanned on the Affymetrix GeneChip scanner, and data was analyzed using the Affymetrix GeneChip software to determine the specifically hybridizing signal for each gene. The differentially expressed genes demonstrated at least a three-fold change in signal when comparing between tissue samples. The differences were all statistically significant (p<0.001)when compared to controls using a T-test.

3. Real Time PCR Analysis

Reverse transcription reactions were performed using up to 3.6 ug mRNA. The RNA was incubated for five minutes at 70° C. and then chilled on ice. A master mix was added containing dNTPs, RT buffer (259 mM Tris-HCl pH 8.3,375 mM KCl, 15 mM MgCl₂), dithiothreitol, random hexamers, RNasin, and reverse transcriptase (Life Technologies, Rockville, Md.). The reactions were incubated for 60 minutes at 37° C., denatured for 5 minutes at 90° C., then chilled on ice for 5 minutes. PCR reactions were performed on ABI Prism® 5700 Sequence Detection System with SYBR green core reagents (PE Applied Biosystem, Foster City, Calif.). All PCR was done at 40 cycles with a pre-incubation period of 50° C. for 2 minutes followed by 95° C. for 10 minutes. Cycling conditions were 95° C. 15 seconds, 55° C. 20 seconds, 75° C. one minute. Some reactions were done with cycling conditions of 95° C. 15 seconds, 60° C. 60 seconds. All data was normalized relative to the signal for the housekeeping gene human hypoxanthine phosphoribosyltransferase I (“HPRT”) (Accession No. BC000578; GI: 12653602) (SEQ ID NO:2), the nucleotide sequence of which is set forth in Table 2, below.

TABLE 2
Human Hypoxanthine Phosphoribosyltransferase I: Nucleotide Sequence
Accession No. BC000578; GI: 12653602

(SEQ ID NO:2)

| | |
|---|---|
| 1 | ggcacgaggc ctcctgagca gtcagcccgc gcgccggccg gctccgttat ggcgacccgc |
| 61 | agccctggcg tcgtgattag tgatgatgaa ccaggttatg accttgattt attttgcata |
| 121 | cctaatcatt atgctgagga tttggaaagg gtgtttattc ctcatggact aattatggac |
| 181 | aggactgaac gtcttgctcg agatgtgatg aaggagatgg gaggccatca cattgtagcc |
| 241 | ctctgtgtgc tcaagggggg ctataaattc tttgctgacc tgctggatta catcaaagca |
| 301 | ctgaatagaa atagtgatag atccattcct atgactgtag attttatcag actgaagagc |
| 361 | tattgtaatg accagtcaac aggggacata aaagtaattg gtggagatga tctctcaact |
| 421 | ttaactggaa agaatgtctt gattgtggaa gatataattg acactggcaa aacaatgcag |
| 481 | actttgcttt ccttggtcag gcagtataat ccaaagatgg tcaaggtcgc aagcttgctg |
| 541 | gtgaaaagga ccccacgaag tgttggatat aagccagact ttgttggatt tgaaattcca |
| 601 | gacaagtttg ttgtaggata tgcccttgac tataatgaat acttcaggga tttgaatcat |
| 661 | gtttgtgtca ttagtgaaac tggaaaagca aaatacaaag cctaagatga gagttcaagt |
| 721 | tgagtttgga aacatctgga gtcctattga catcgccagt aaaattatca atgttctagt |
| 781 | tctgtggcca tctgcttagt agagcttttt gcatgtatct tctaagaatt ttatctgttt |
| 841 | tgtactttag aaatgtcagt tgctgcattc ctaaactgtt tatttgcact atgagcctat |
| 901 | agactatcag ttccctttgg gcggattgtt gtttaacttg taaatgaaaa aattctctta |
| 961 | aaccacagca ctattgagtg aaacattgaa ctcatatctg taagaaataa agagaagata |
| 1021 | tattagtttt ttaattggta ttttaatttt tatatatgca ggaaagaata gaagtgattg |
| 1081 | aatattgtta attataccac cgtgtgttag aaaagtaaga agcagtcaat tttcacatca |
| 1141 | aagacagcat ctaagaagtt ttgttctgtc ctggaattat tttagtagtg tttcagtaat |
| 1201 | gttgactgta ttttccaact tgttcaaatt attaccagtg aatctttgtc agcagttccc |
| 1261 | ttttaaatgc aaatcaataa attcccaaaa atttaaaaaa aaaaaaaaaa aaaaaa |

Primer sets were as follows:

HPRT:

| | | |
|---|---|---|
| Forward: | GGTATACTGCCTGACCAAGG | (SEQ ID NO:3) |
| Reverse: | CGAGATGTGATGAAGGAGATGG | (SEQ ID NO:4) |

GI:4070352 Scavenger Receptor with C-Type Lectin Type I

| | | | |
|---|---|---|---|
| Forward | 246-266 | GCACCTTGGGAGTTATGTTT | (SEQ ID NO:5) |
| Reverse | 393-413 | ATGACTGTACTTGGCATCAG | (SEQ ID NO:6) |

### EXAMPLE 1

### Upregulated Genes and Downregulated Genes in OA

As shown in some of the Figures referenced below, start and stop codons are bolded for clarity.

1. Maternally Expressed-3 Expression

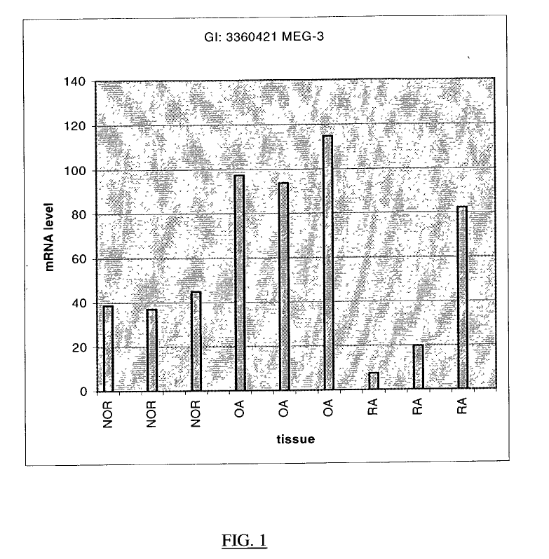

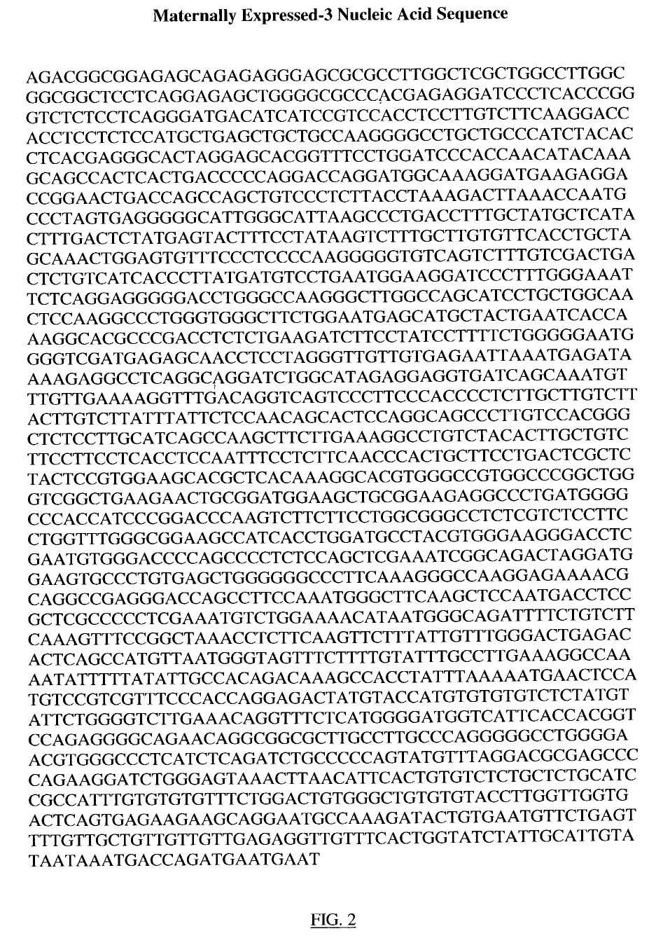

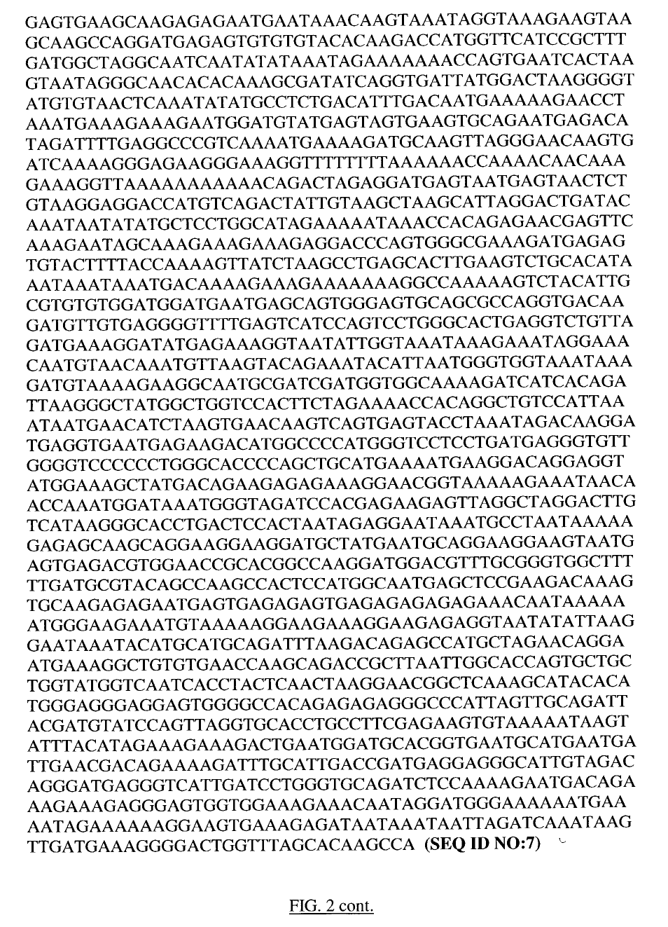

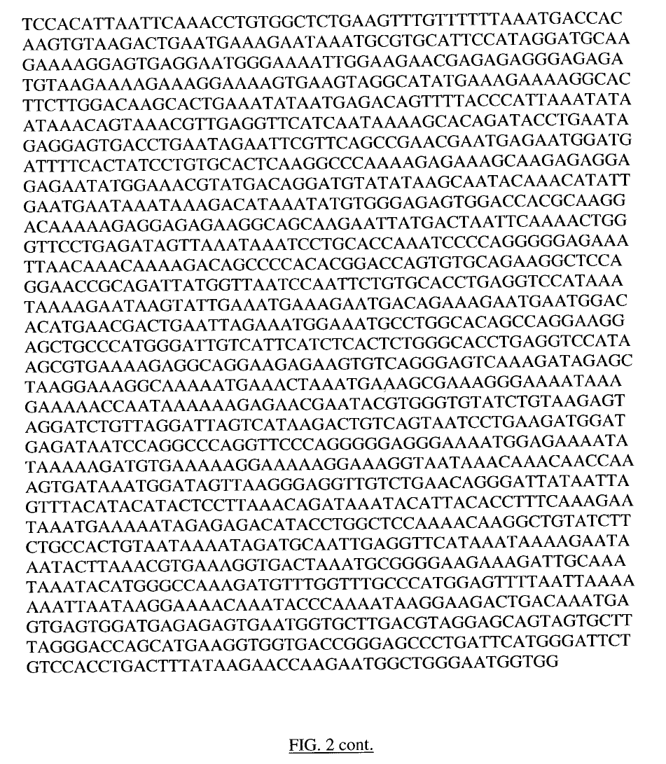

Using the materials and methods described hereinabove, increases in expression of Maternally Expressed-3 (MEG-3) (GI:3360421) were detected in the OA synovium. This increased expression is shown in the microarray data in FIG. 1. The polynucleotide sequence (SEQ ID NO:7) of MEG-3 is shown in FIG. 2.

MEG-3 has no obvious open reading frame leaving its function unclear (Miyoshi, et al., Genes Cells 5:211-220 (2000)).

2. Hypothetical Protein FLJ20424 Expression

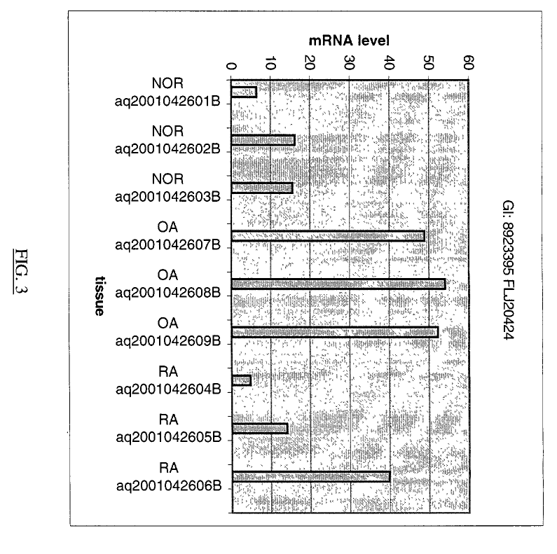

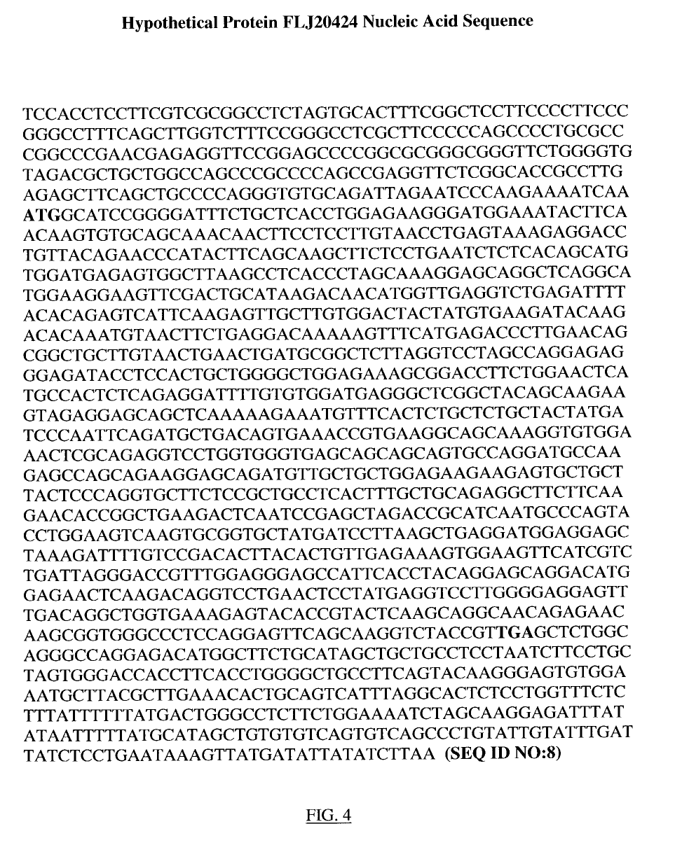

Using the materials and methods described hereinabove, increases in expression of Hypothetical Protein FLJ20424 (GI:8923395; GI:10438720) were detected in the OA synovium. This increased expression is shown in the microarray data in FIG. 3. The polynucleotide sequence (SEQ ID NO:8) and amino acid sequence (SEQ ID NO:9) of Hypothetical Protein FLJ20424 are shown in FIGS. 4 and 5, respectively.

3. Scavenger Receptor with C-Type Lectin Type 1 Expression

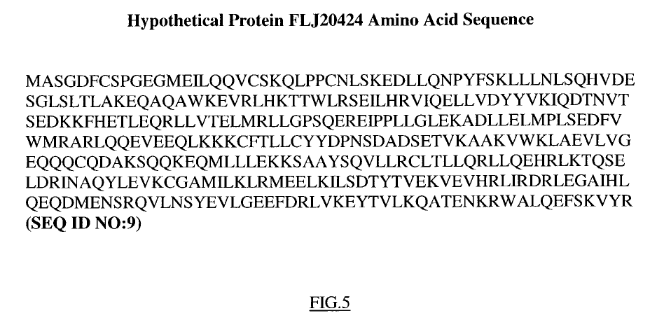

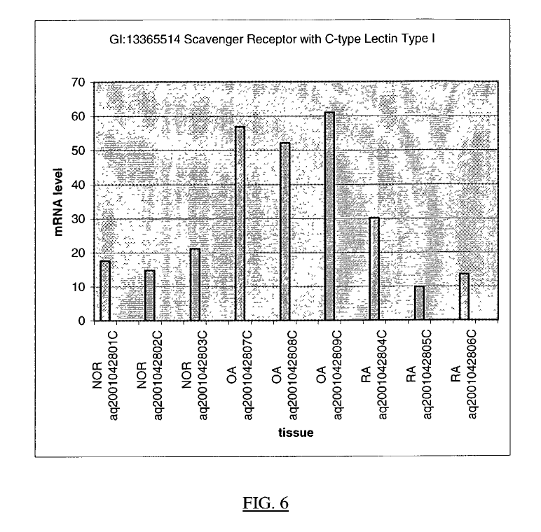

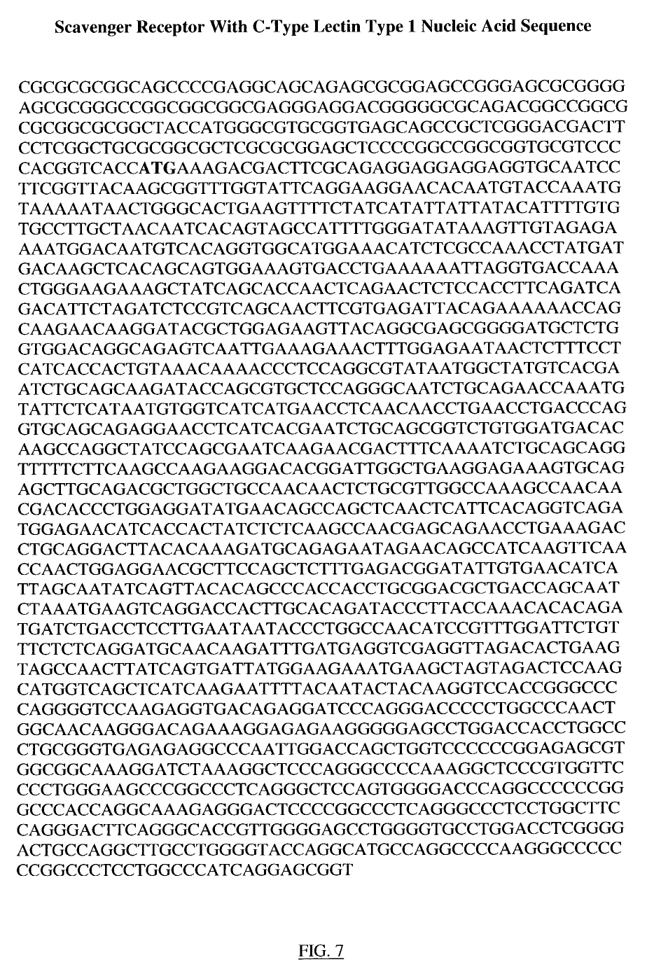

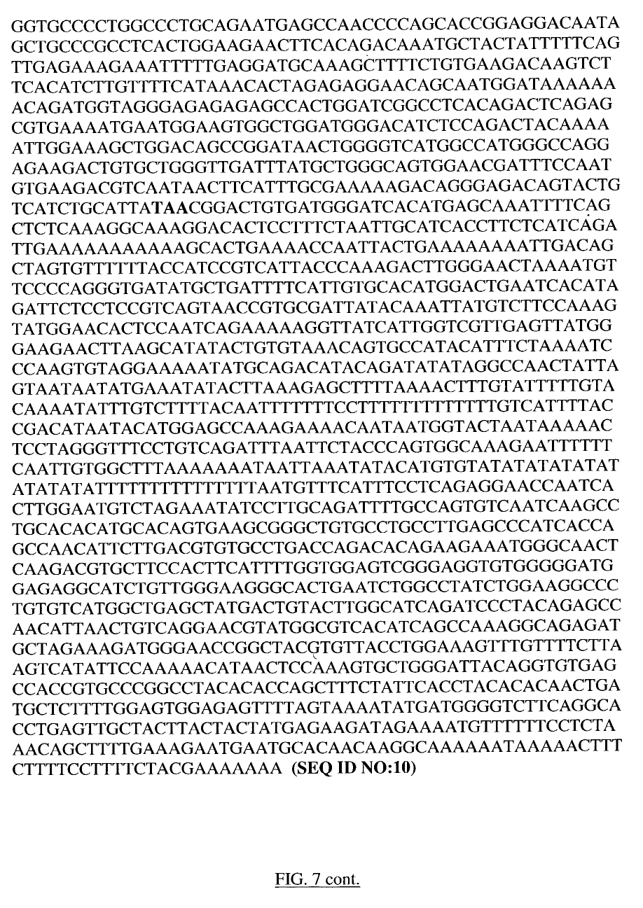

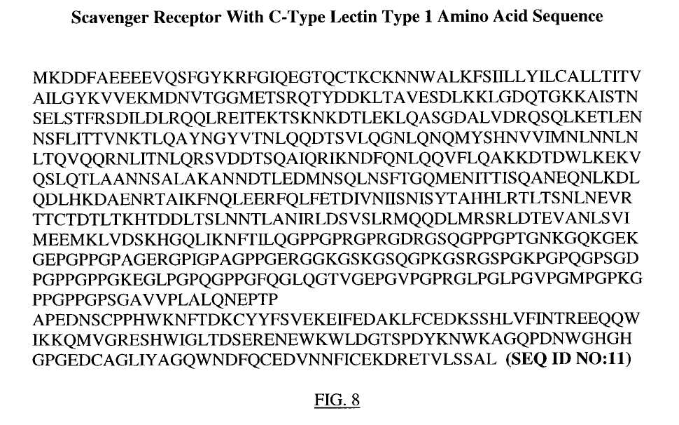

Using the materials and methods described hereinabove, increases in expression of Scavenger Receptor With C-Type Lectin Type 1 (GI:13365514; GI:13365515)) were detected in the OA synovium. This increased expression is shown in the microarray data in FIG. 6. The polynucleotide sequence (SEQ ID NO:10) and amino acid sequence (SEQ ID NO:11) of Scavenger Receptor With C-Type Lectin Type 1 are shown in FIGS. 7 and 8, respectively.

Using the materials and methods described hereinabove, Real Time PCR was conducted to quantify the expression of Scavenger Receptor With C-Type Lectin Type 1 in the OA synovium, the results of which are set forth in Table 3, below. As used in Table 3, “RA” stands for “Rheumatoid Arthritis”.

TABLE 3
Scavenger Receptor With C-Type Lectin Type 1: Real Time PCR Results

| | Expression | |
|---|---|---|
| | Level | T test |
| Normal | 1 | |
| OA | 4.45 | 0.020 |
| RA | 0.85 | 0.805 |

Scavenger Receptor With C-Type Lectin Type 1 is a bacteria-binding receptor that may play a role in host defense (Nakamura, et al., Biochem. Biophys. Res. Comm. 280:1028-1035 (2001)).

4. Tetranectin Expression

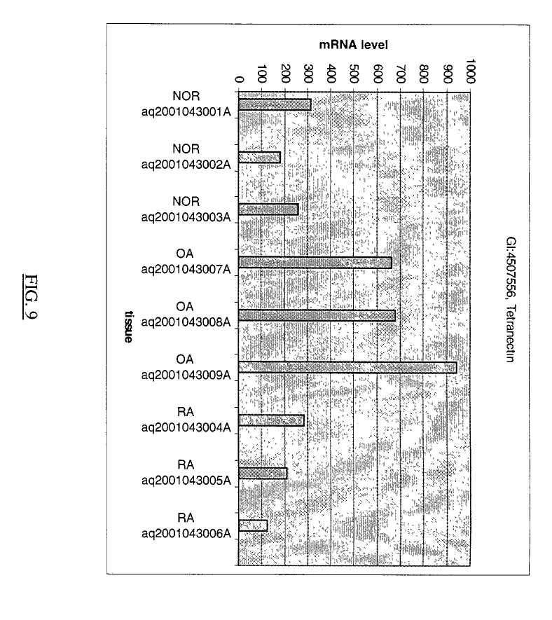

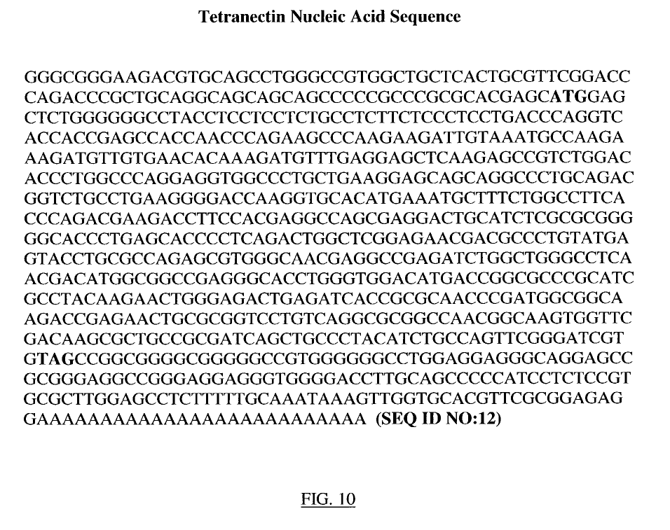

Using the materials and methods described hereinabove, increases in expression of Tetranectin (GI:4507556; GI:4507557) were detected in the OA synovium. This increased expression is shown in the microarray data in FIG. 9. The polynucleotide sequence (SEQ ID NO:12) and amino acid sequence (SEQ ID NO:13) of Tetranectin are shown in FIGS. 10 and 11, respectively.

Tetranectin is a plasminogen-binding, homotrimeric protein belonging to the C-type lectin family of proteins (Clemmensen, et al., Eur. J. Biochem. 156:327-333 (1986)). Levels of Tetranectin have been found to be elevated in synovial fluid from rheumatoid arthritis patients as compared to synovial fluid from OA and seronegative spondylarthritis patients (Kamper, et al., Clin. Rheumatol. 17:318-324 (1998)).

5. Retinoblastoma Binding Protein 6 Expression

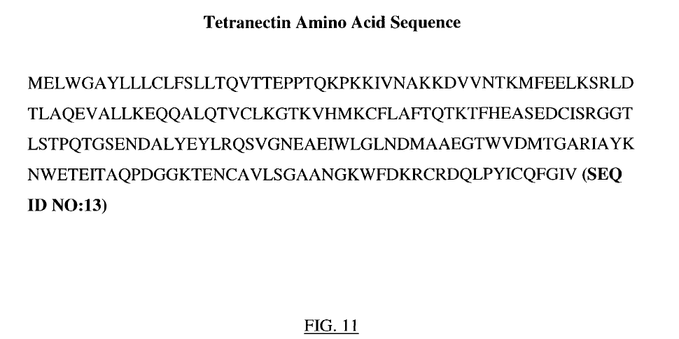

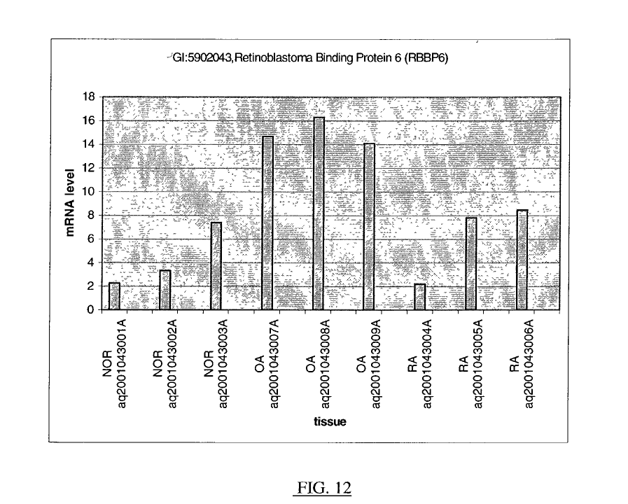

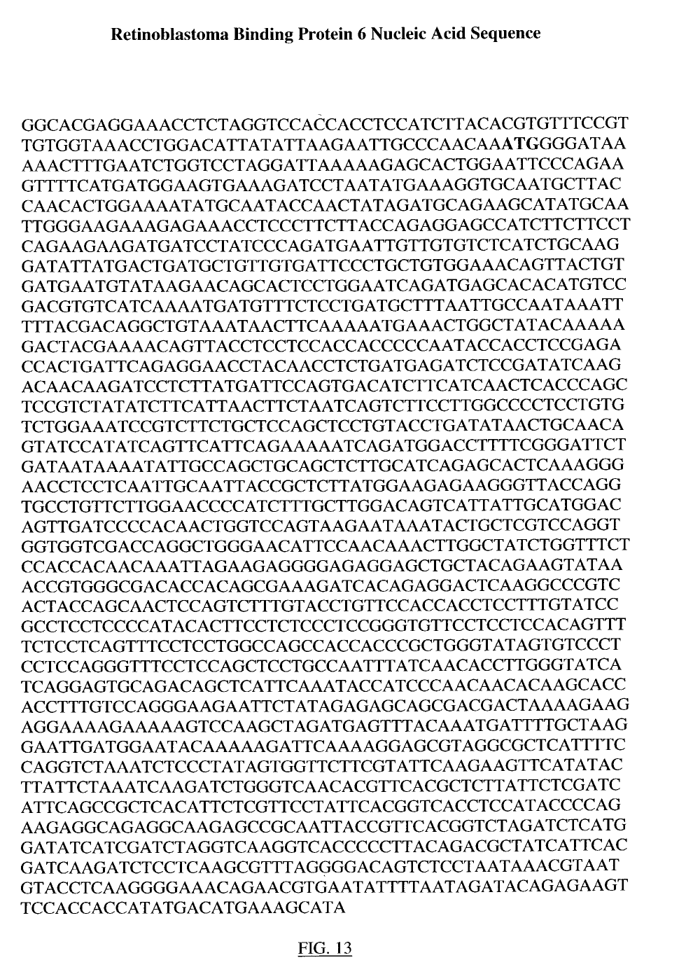

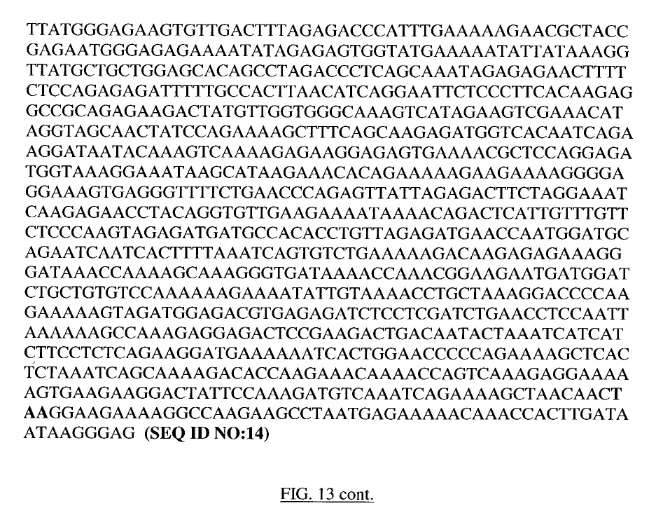

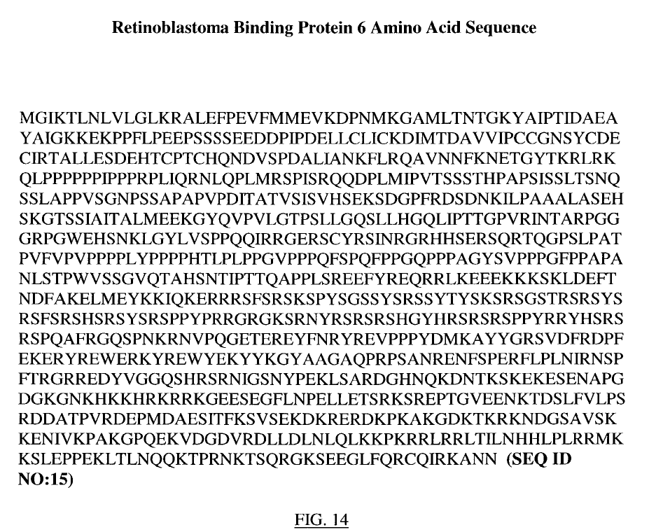

Using the materials and methods described hereinabove, increases in expression of Retinoblastoma Binding Protein 6 (RBBP6) (GI:5902043; GI:5902044)) were detected in the OA synovium. This increased expression is shown in the microarray data in FIG. 12. The polynucleotide sequence (SEQ ID NO:14) and amino acid sequence (SEQ ID NO:15) of RBBP6 are shown in FIGS. 13 and 14, respectively.

RBBP6 is a novel retinoblastoma binding protein that interacts with underphosphorylated but not phosphorylated retinoblastoma (Sakai, et al., Genomics 30:98-101 (1995)).

6. Homo Sapiens ATP Synthase H+ Transporting, Mitochondrial F0 Complex, Subunit G Expression

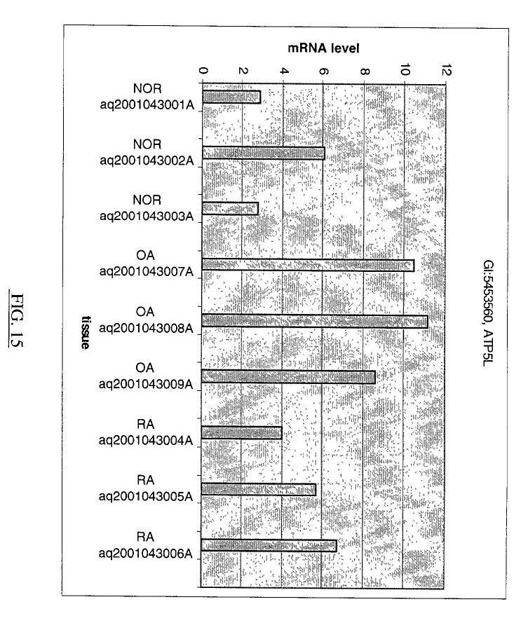

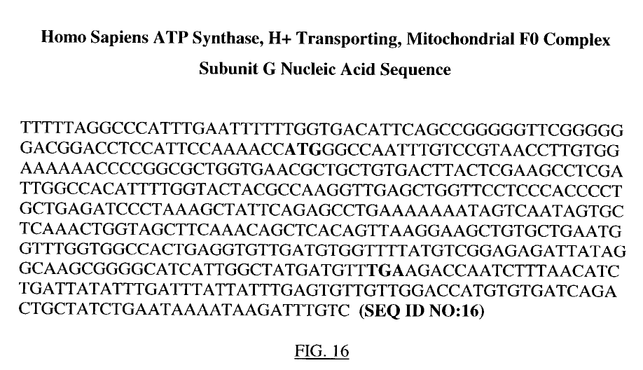

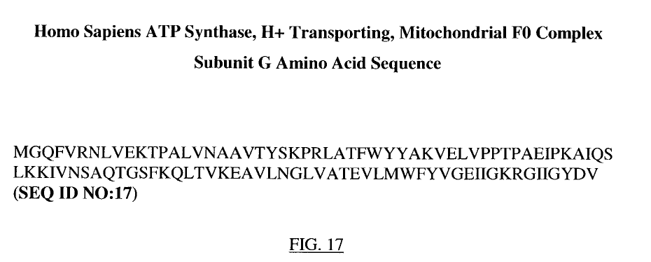

Using the materials and methods described hereinabove, increases in expression of Homo Sapiens ATP Synthase, H+ Transporting, Mitochondrial F0 complex, Subunit G Expression (ATP5L) (GI:5453560; GI:5453561) were detected in the OA synovium. This increased expression is shown in the microarray data in FIG. 15. The polynucleotide sequence (SEQ ID NO:16) and amino acid sequence (SEQ ID NO:17) of ATP5L are shown in FIGS. 16 and 17, respectively.

ATP5L is one of the chains of the nonenzymatic component of the mitochondrial ATPase complex.

7. Homo Sapiens Proline Synthetase Co-Transcribed (Bacterial Homolog Expression

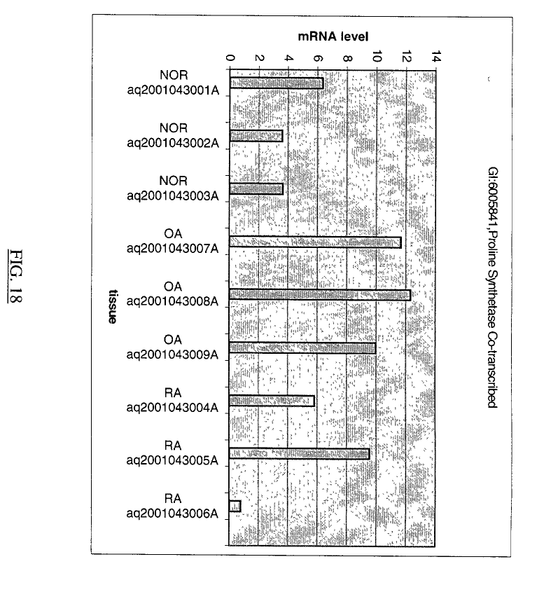

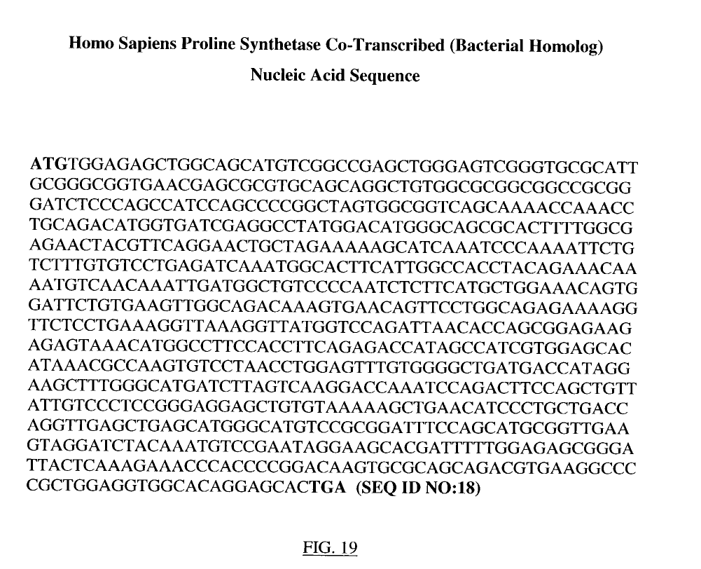

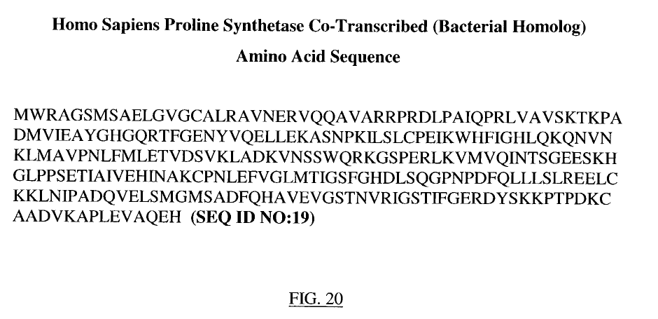

Using the materials and methods described hereinabove, significant increases in expression of Homo Sapiens Proline Synthetase Co-Transcribed (Bacterial Homolog) (GI:6005841; GI:6005842) were detected in the OA synovium. This increased expression is shown in the microarray data in FIG. 18. The polynucleotide sequence (SEQ ID NO:18) and amino acid sequence (SEQ ID NO:19) of Homo Sapiens Proline Synthetase Co-Transcribed (Bacterial Homolog) are shown in FIGS. 19 and 20, respectively.

The function of this protein has not been characterized.

8. NADH Dehydrogenase (Ubiquinone) 1 Alpha Subcomplex, 2 Expression

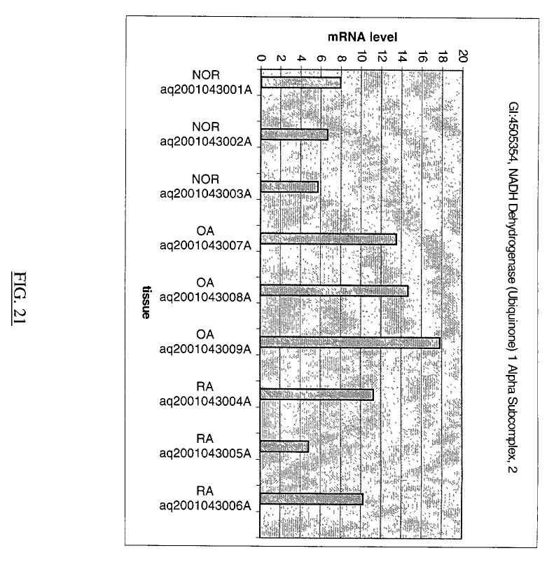

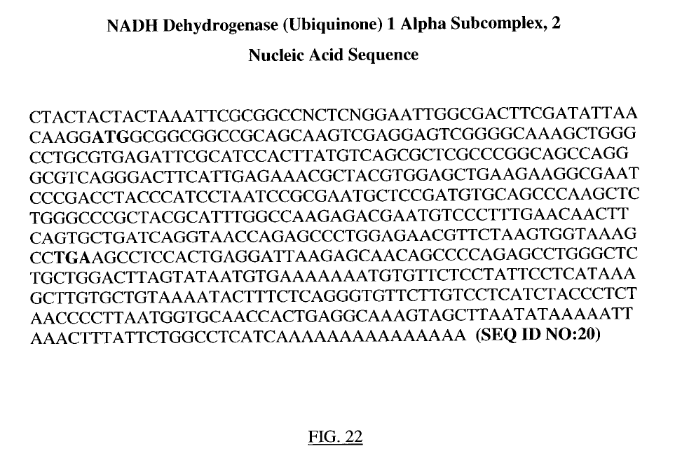

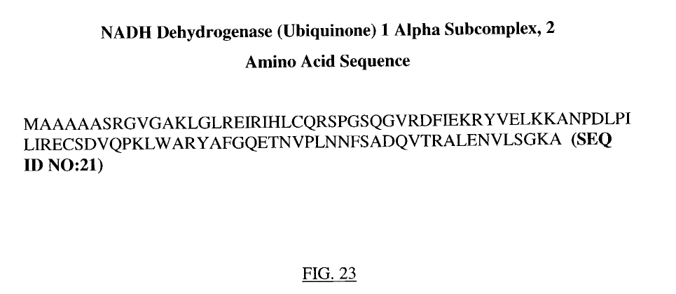

Using the materials and methods described hereinabove, increases in expression of NADH Dehydrogenase (Ubiquinone) 1 Alpha Subcomplex, 2 (GI:4505354; GI:4505355) were detected in the OA synovium. This increased expression is shown in the microarray data in FIG. 21. The polynucleotide sequence (SEQ ID NO:20) and amino acid sequence (SEQ ID NO:21) of NADH Dehydrogenase (Ubiquinone) 1 Alpha Subcomplex, 2 are shown in FIGS. 22 and 23, respectively.

This protein is a subunit of the mitochondrial complex that transfers electrons from NADH to the respiratory chain.

9. Protein C Receptor, Endothelial Expression

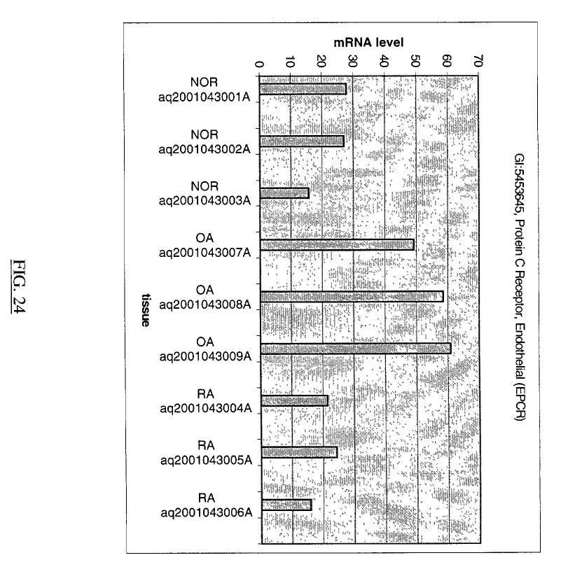

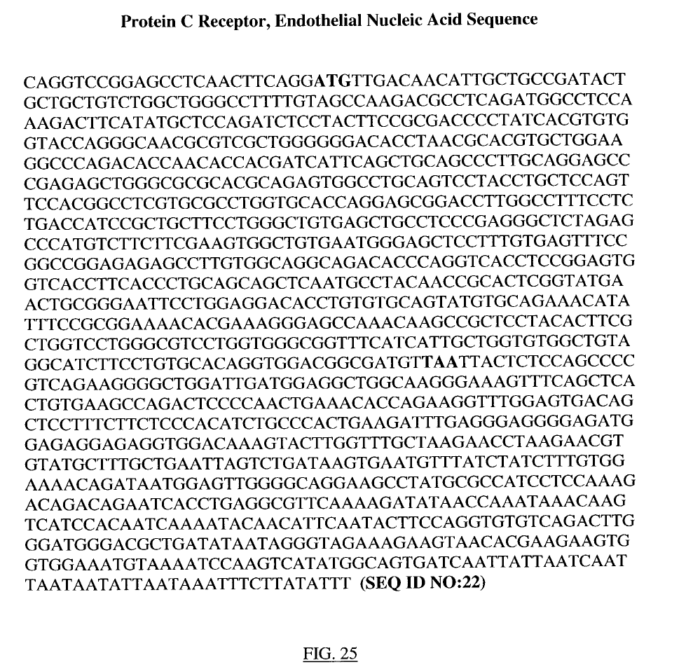

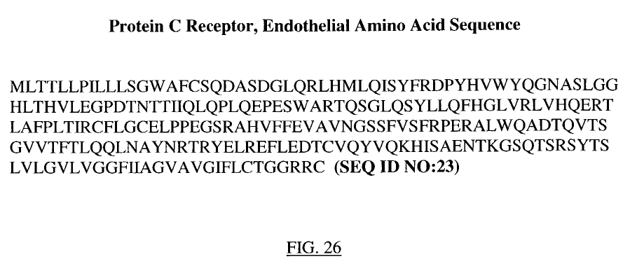

Using the materials and methods described hereinabove, increases in expression of Protein C receptor, Endothelial (EPCR) (GI:5453545; GI:5453646) were detected in the OA synovium. This increased expression is shown in the microarray data in FIG. 24. The polynucleotide sequence (SEQ ID NO:22) and amino acid sequence (SEQ ID NO:23) of Protein C receptor, Endothelial (EPCR) are shown in FIGS. 25 and 26 respectively.

EPCR binds activated Protein C and augments Protein C activation by the thrombin-thrombomodulin complex (Taylor, et al., Blood 97:1685-1688 (2001)).

10. Beta-Galactose-3-O-Sulfotransferase, 4 Expression

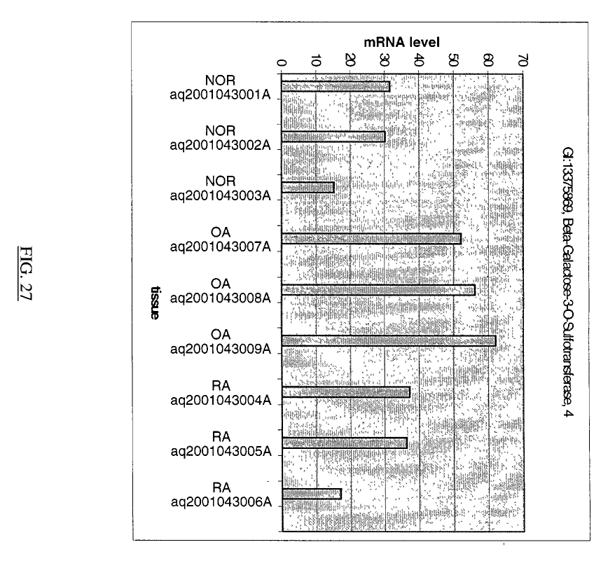

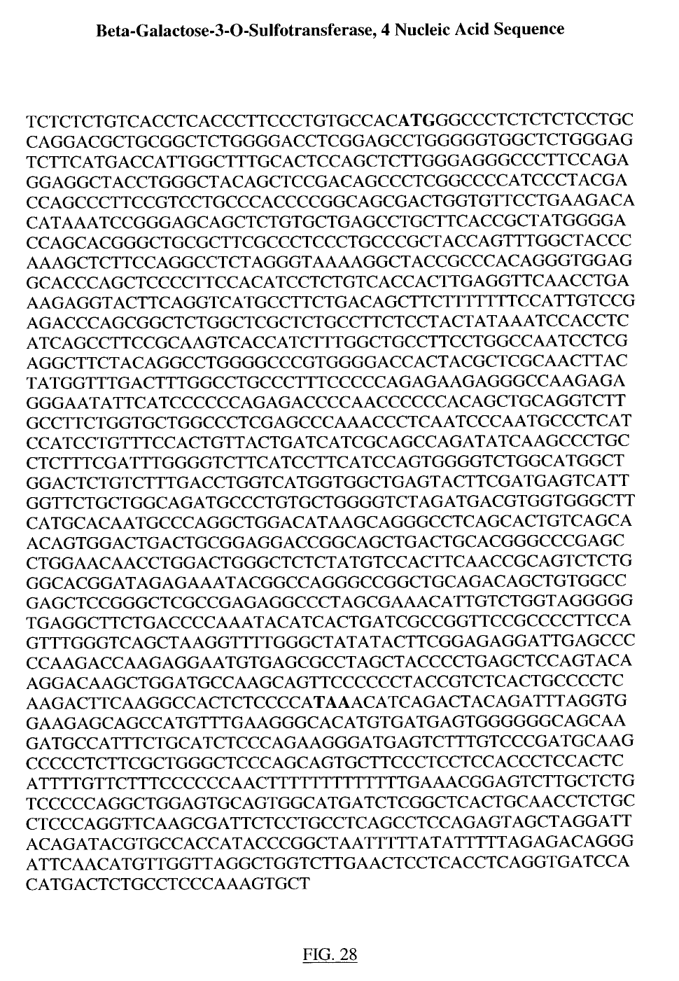

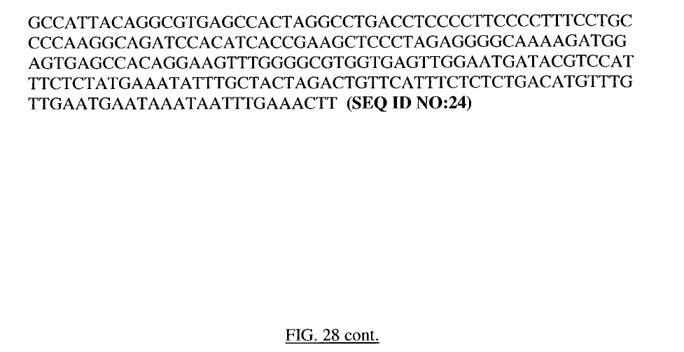

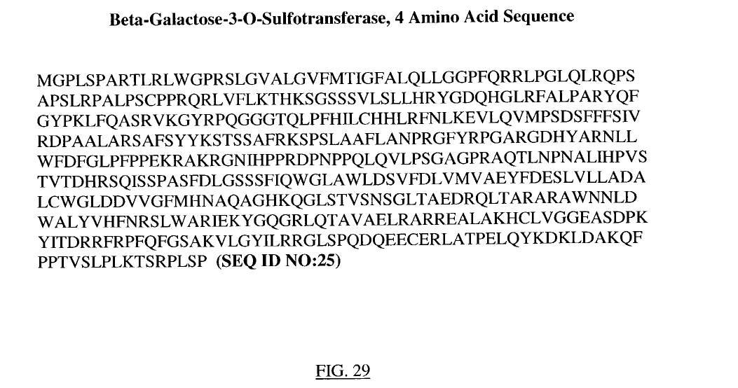

Using the materials and methods described hereinabove, increases in expression of Beta-Galactose-3-O-Sulfotransferase, 4 (Gal3ST-4) (GI: 13375869; GI: 13375870) were detected in the OA synovium. This increased expression is shown in the microarray data in FIG. 27. The polynucleotide sequence (SEQ ID NO:24) and amino acid sequence (SEQ ID NO:25) of Beta-Galactose-3-O-Sulfotransferase, 4 are shown in FIGS. 28 and 29, respectively.

Gal3ST-4 catalzyes the transfer of sulfate to the C-3′ position of Galβ1-3GalNAc in O-linked glycans (Seko, et al., J. Biol. Chem. 276:25697-25704 (2001)).

11. Homo Sapiens RPB5-Mediated Protein Expression

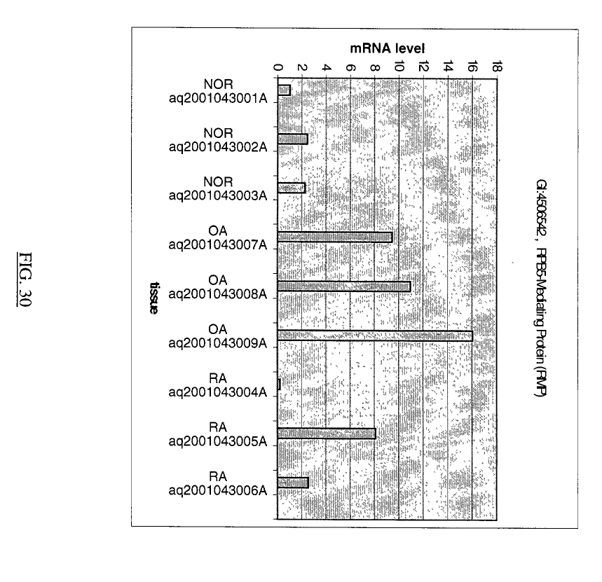

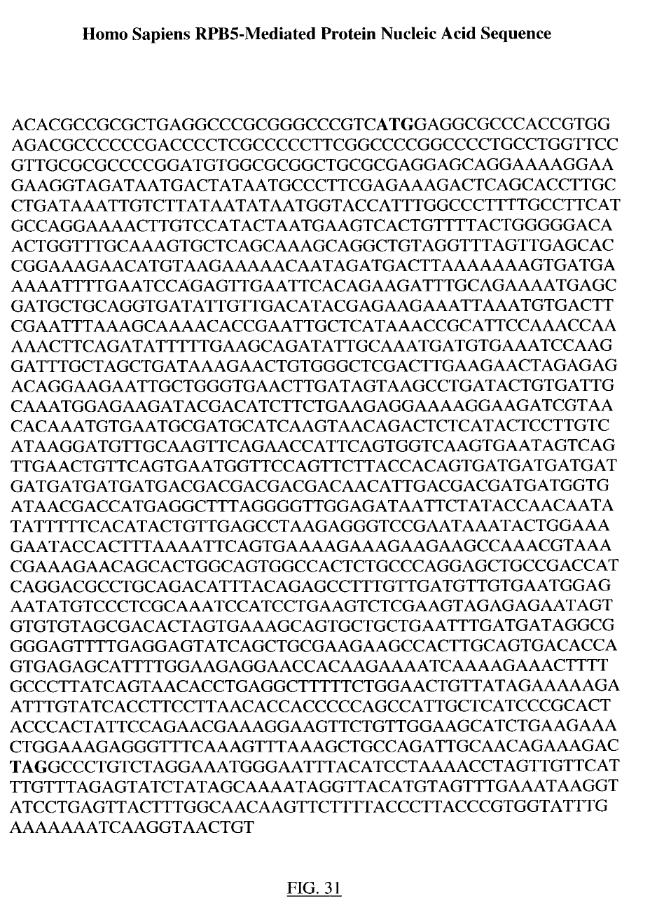

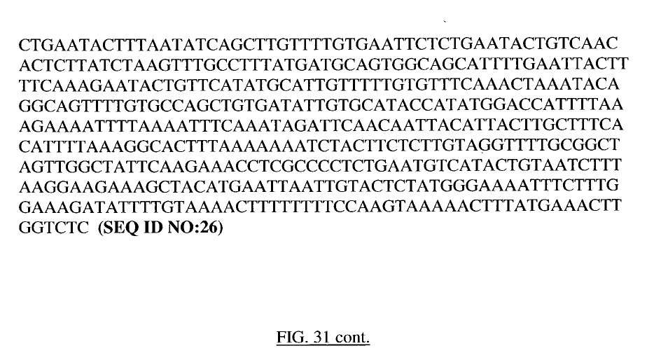

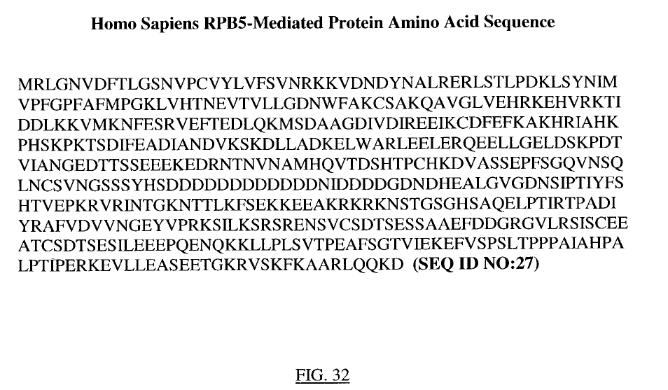

Using the materials and methods described hereinabove, increases in expression of Homo Sapiens RPB5-Mediated Protein (RMP) (GI:4506542; GI:4506543) were detected in the OA synovium. This increased expression is shown in the microarray data in FIG. 30. The polynucleotide sequence (SEQ ID NO:26) and amino acid sequence (SEQ ID NO:27) of RMP are shown in FIGS. 31 and 32, respectively.

RMP was isolated by its ability to interact with RNA polymerase II subunit 5, suggesting that it may regulate transcription (Dorjsuren, et al., Mol. Cell Biol. 18:7546-7555 (1998)).

12. GI:4896280 Expression

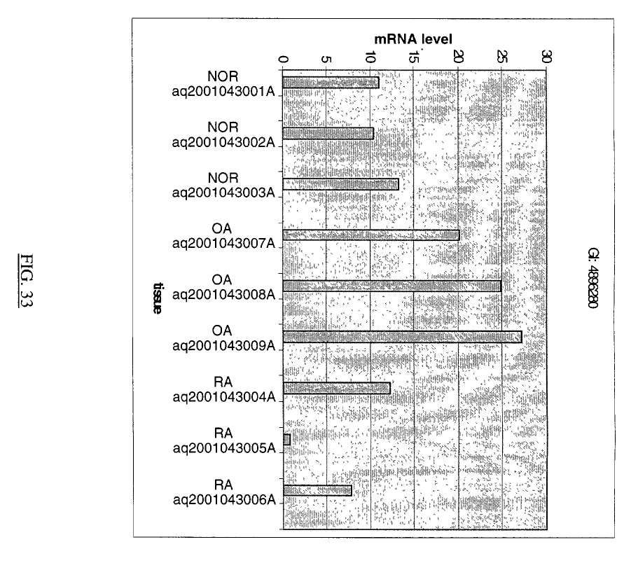

Using the materials and methods described hereinabove, increases in expression of the polynucleotide having the sequence set forth at GI:4896280 were detected in the OA synovium. This increased expression is shown in the microarray data in FIG. 33. The nucleic acid sequence (SEQ ID NO:28) of this polynucleotide is shown in FIG. 34.

13. Homo Sapiens Hypothetical Protein FLJ123399 Expression

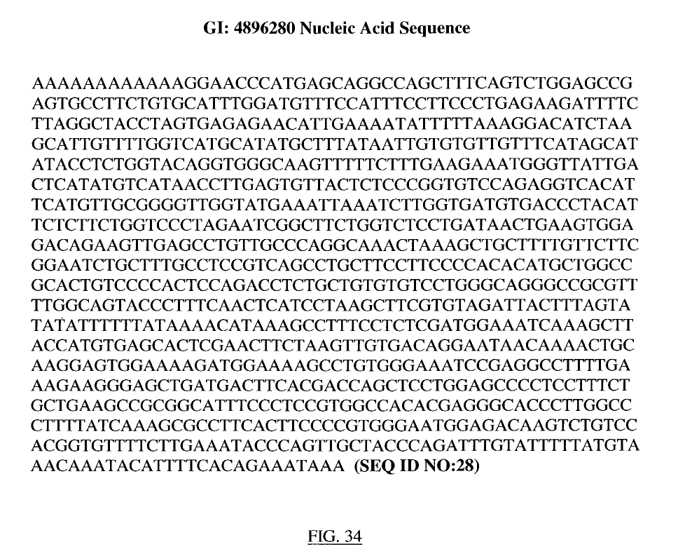

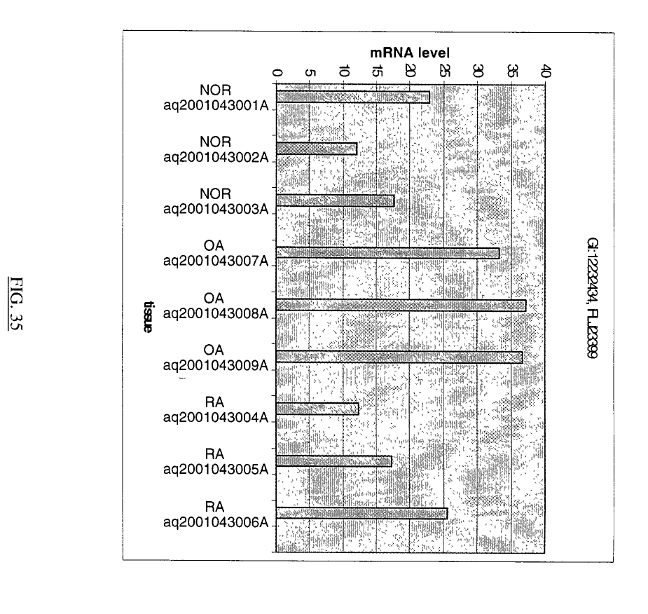

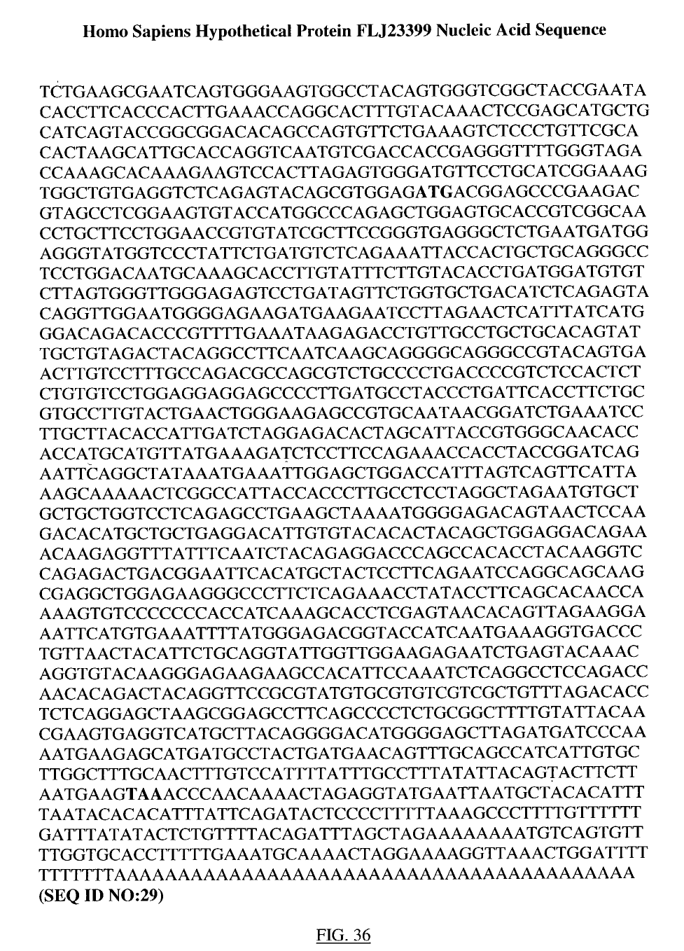

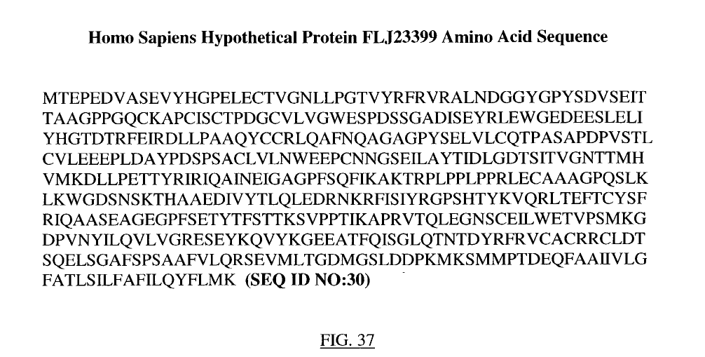

Using the materials and methods described hereinabove, increases in expression of Homo Sapiens Hypothetical Protein FLJ23399 (GI: 12232434) were detected in the OA synovium. This increased expression is shown in the microarray data in FIG. 35. The polynucleotide sequence (SEQ ID NO:29) and amino acid sequence (SEQ ID NO:30) of Homo Sapiens Hypothetical Protein FLJ23399 are shown in FIGS. 36 and 37, respectively.

The predicted protein sequence of FLJ23399 contains four fibronectin type III domains. Proteins containing this domain have been shown to bind DNA, heparin, and cell surfaces.

14. Homo sapiens Hypothetical Protein FL122662 Expression

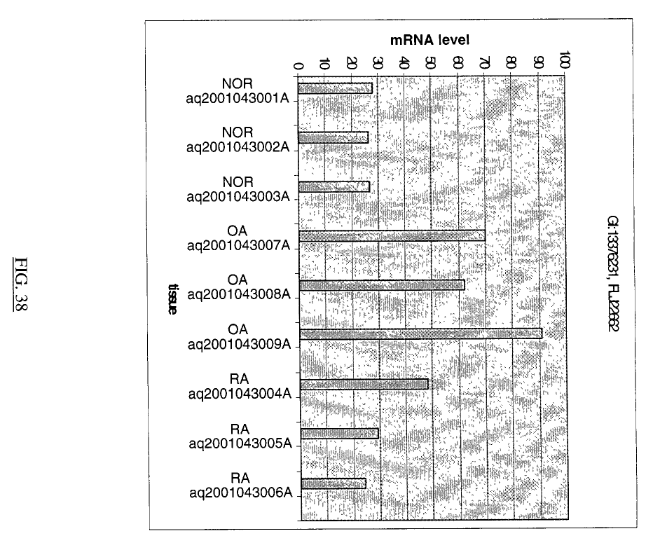

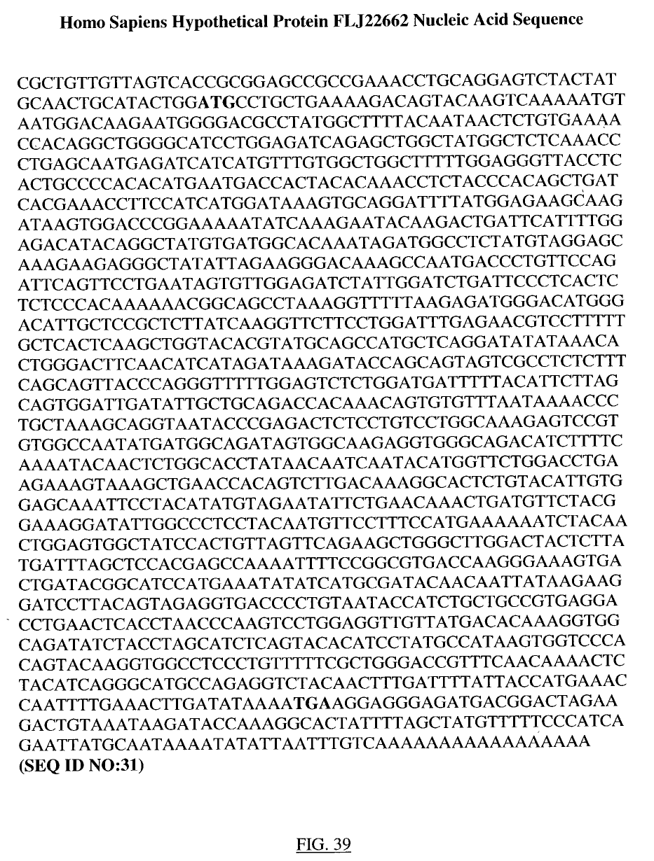

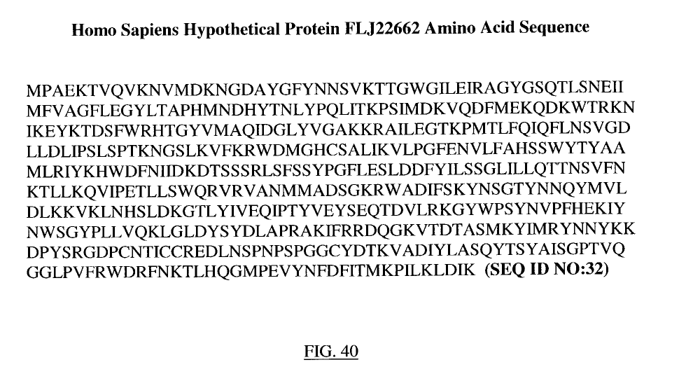

Using the materials and methods described hereinabove, increases in expression of Homo sapiens hypothetical protein FLJ22662 (GI:13376231) were detected in the OA synovium. This increased expression is shown in the microarray data in FIG. 38. The polynucleotide sequence (SEQ ID NO:31) and amino acid sequence (SEQ ID NO:32) of Homo sapiens hypothetical protein FLJ22662 are shown in FIGS. 39 and 40, respectively.

15. IL-22; Transforming Growth Factor Beta-9 Expression

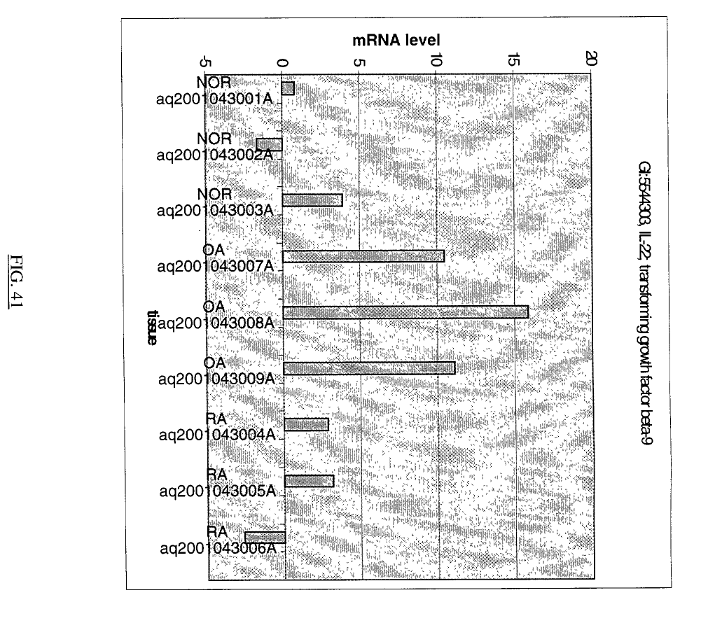

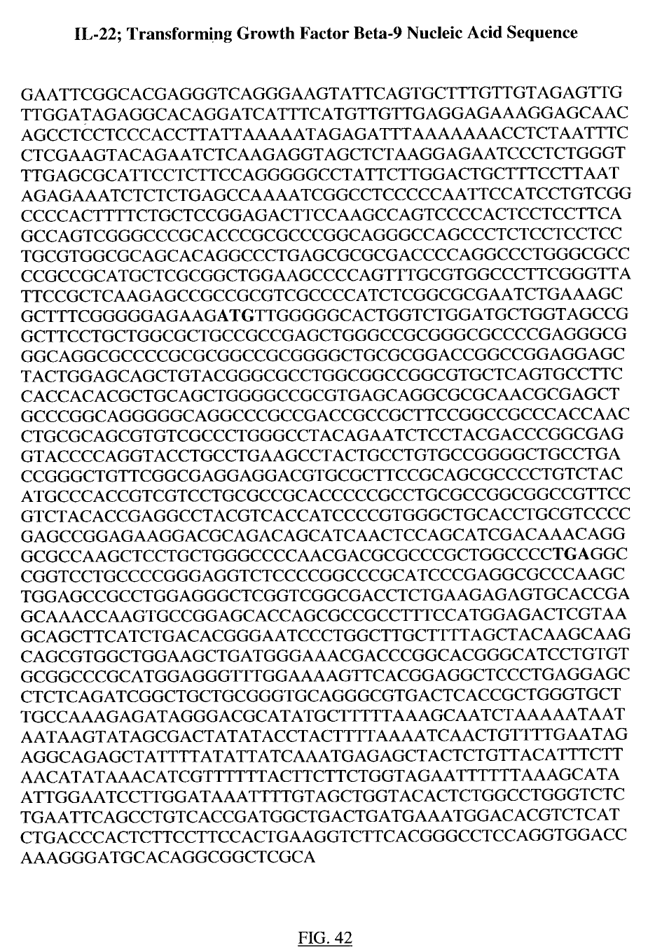

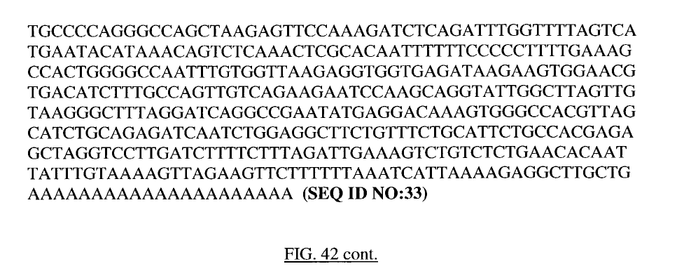

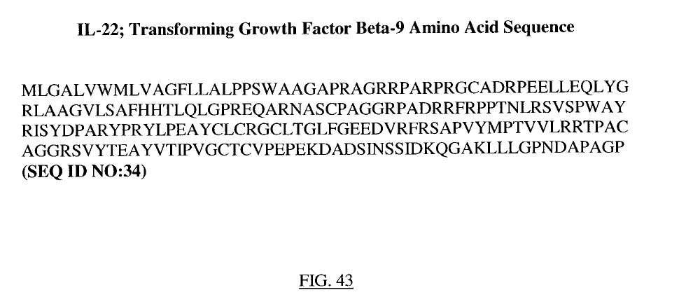

Using the materials and methods described hereinabove, increases in expression of IL-22; Transforming Growth Factor Beta-9 (GI:5544303) were detected in the OA synovium. This increased expression is shown in the microarray data in FIG. 41. The polynucleotide sequence (SEQ ID NO:33) and amino acid sequence (SEQ ID NO:34) of IL-22; Transforming Growth Factor Beta-9 are shown in FIGS. 42 and 43, respectively.

IL-22 is an IL-10 homolog produced by T cells. It induces the production of acute phase reactants in vitro and in vivo (Xu, et al., Proc. Natl. Acad. Sci. USA 98:951 1-9516 (2001)).

16. NADH Dehydrogenase (Ubiquinone) 1 Alpha Subcomplex, 4 Expression

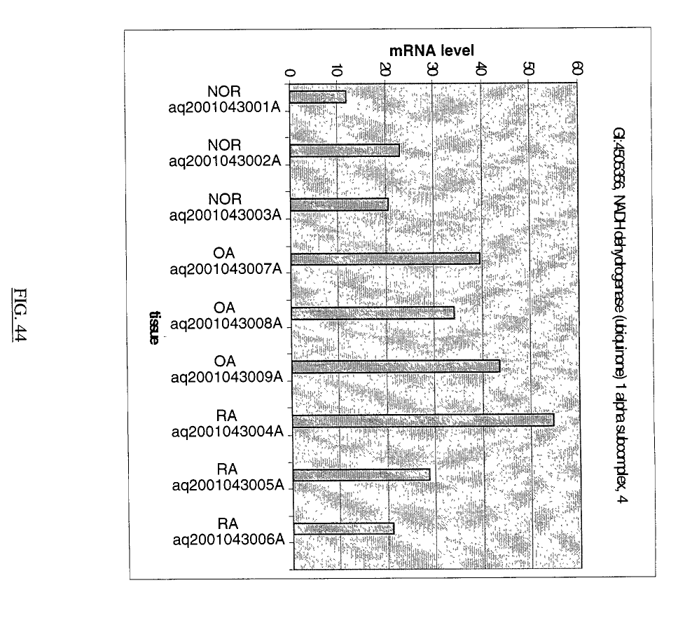

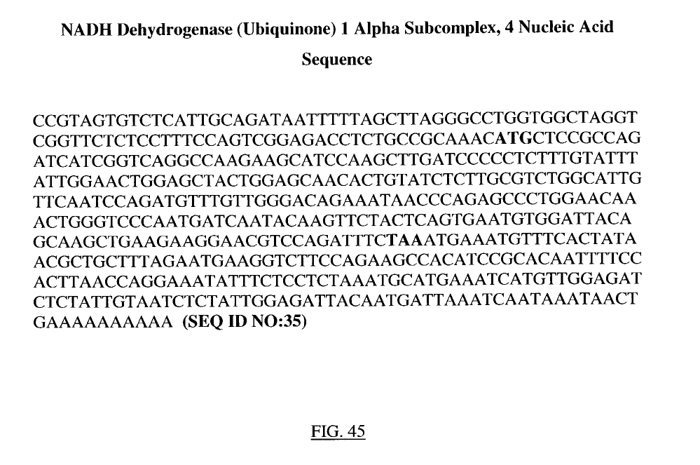

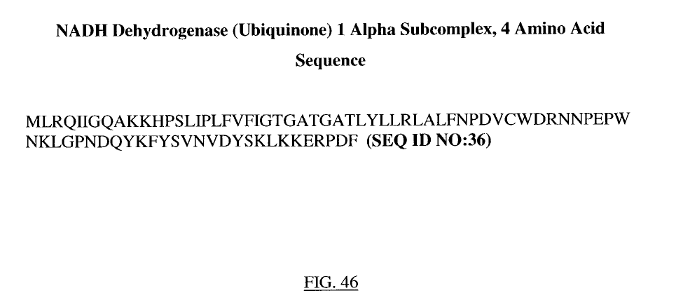

Using the materials and methods described hereinabove, increases in expression of NADH Dehydrogenase (Ubiquinone) 1 Alpha Subcomplex, 4 were detected in the OA synovium. This increased expression is shown in the microarray data in FIG. 44. The polynucleotide sequence (SEQ ID NO:35) and amino acid sequence (SEQ ID NO:36) of NADH Dehydrogenase (Ubiquinone) 1 Alpha Subcomplex, 4 are shown in FIGS. 44 and 46, respectively.

This protein is a component of the mitochondrial complex that transfers electrons from NADH to the respiratory chain.

What is claimed is:

1. An assay for identifying a compound that modulates the activity of a gene associated with osteoarthritis, comprising:
(1) providing a cell expressing a gene associated with osteoarthritis, wherein the nucleic acid sequence of said gene associated with osteoarthritis is at least 95% identical to a nucleic acid sequence selected from the group consisting of SEQ ID NO:7, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:31, SEQ ID NO:33, and SEQ ID NO:35;
(2) contacting said cell expressing said gene associated with osteoarthritis with a test compound; and
(3) determining whether said test compound modulates the activity of said gene associated with osteoarthritis.

2. The assay of claim 1, wherein said assay is a cell-based assay.

3. The assay of claim 1, wherein said assay is a cell-free assay.

4. The assay of claim 3, wherein said cell-free assay is a ligand-binding assay.

5. The assay of claim 1, wherein said test compound modulates the activity of said gene associated with osteoarthritis.

6. The assay of claim 1, wherein said test compound is an antagonist of a gene associated with osteoarthritis.

7. The assay of claim 1, wherein said test compound is an agonist of a gene associated with osteoarthritis.

8. The assay of claim 1, wherein said test compound binds to said gene associated with osteoarthritis.

9. The assay of claim 1, wherein said assay is useful for identifying compounds which are useful for the treatment of osteoarthritis.

10. An assay for identifying a compound that modulates the activity of a protein associated with osteoarthritis, comprising:
(1) providing a cell expressing a gene associated with osteoarthritis, wherein said gene encodes a polypeptide having an amino acid sequence which is at least 95% identical to an amino acid sequence selected from the group consisting of SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:30, SEQ ID NO:32, SEQ ID NO:34, and SEQ ID NO:36;
(2) contacting said cell expressing said gene associated with osteoarthritis with a test compound; and
(3) determining whether said test compound modulates the activity of said protein associated with osteoarthritis.

11. The assay of claim 10, wherein said assay is a cell-based assay.

12. The assay of claim 10, wherein said assay is a cell-free assay.

13. The assay of claim 12, wherein said cell-free assay is a ligand-binding assay.

14. The assay of claim 10, wherein said test compound modulates the activity of said polypeptide associated with osteoarthritis.

15. The assay of claim 10, wherein said test compound is an antagonist of a polypeptide associated with osteoarthritis.

16. The assay of claim 10, wherein said test compound is an agonist of a polypeptide associated with osteoarthritis.

17. The assay of claim 10, wherein said test compound binds to said polypeptide associated with osteoarthritis.

18. The assay of claim 10, wherein said assay is useful for identifying compounds which are useful for the treatment of osteoarthritis.

19. A method for the treatment of osteoarthritis, comprising:
(1) identifying a patient suffering from osteoarthritis; and
(2) administering to said patient a therapeutically effective amount of a modulator of a gene associated with osteoarthritis,
wherein said gene associated with osteoarthritis has a nucleic acid sequence which is at least 95% identical to a nucleic acid sequence selected from the group consisting of SEQ ID NO:7, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:29, SEQ ID NO:31, SEQ ID NO:33, and SEQ ID NO:35.

20. The method of claim 19, wherein said patient is identified as suffering from osteoarthritis by measuring the expression level of said gene associated with osteoarthritis in said patient.

21. The method of claim 19, wherein said modulator is an antagonist of a gene associated with osteoarthritis.

22. A method for the treatment of osteoarthritis, comprising:
(1) identifying a patient suffering from osteoarthritis; and
(2) administering to said patient suffering from osteoarthritis a therapeutically effective amount of a modulator of a polypeptide associated with osteoarthritis,
wherein said polypeptide associated with osteoarthritis has an amino acid sequence which is at least 95% identical to an amino acid sequence selected from the group consisting of SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:30, SEQ ID NO:32, SEQ ID NO:34, and SEQ ID NO:36.

23. The method of claim 22, wherein said patient is identified as suffering from osteoarthritis by measuring the expression level of said polypeptide associated with osteoarthritis.

23. The method of claim 22, wherein said modulator is an antagonist of a polypeptide associated with osteoarthritis.

24. A method for the prevention of osteoarthritis, comprising:
(1) identifying a patient at risk for osteoarthritis; and
(2) administering to said patient at risk for osteoarthritis a therapeutically effective amount of a modulator of a gene associated with osteoarthritis,
wherein said gene associated with osteoarthritis has a nucleic acid sequence which is at least 95% identical to a nucleic acid sequence selected from the group consisting of SEQ ID NO:7, SEQ ID NO:8, SEQ ID NO:10, SEQ ID NO:12, SEQ ID NO:14, SEQ ID NO:16, SEQ ID NO:18, SEQ ID NO:20, SEQ ID
